(19) United States
(12) Patent Application Publication
(10) Pub. No.: US 2024/0316028 A1
(43) Pub. Date: Sep. 26, 2024

(54) OPHTHALMIC CREAM FORMULATIONS AND THEIR USES

(71) Applicant: Glaukos Corporation, Aliso Viejo, CA (US)

(72) Inventors: Xiaojun Michael Liu, Lake Forest, CA (US); James Jane-Guo Shiah, Irvine, CA (US)

(21) Appl. No.: 18/606,217

(22) Filed: Mar. 15, 2024

Related U.S. Application Data
(60) Provisional application No. 63/452,633, filed on Mar. 16, 2023.
(60) Provisional application No. 63/605,955, filed on Dec. 4, 2023.
(60) Provisional application No. 63/549,789, filed on Feb. 5, 2024.

(51) Int. Cl. A61K 31/46; A61K 9/00; A61K 9/06; A61K 31/407; A61K 45/06; A61K 47/10; A61K 47/14; A61K 47/18; A61K 47/22; A61K 47/26; A61K 47/32; A61K 47/44

(52) U.S. Cl. CPC A61K 31/46; A61K 9/0014; A61K 9/0048; A61K 9/06; A61K 31/407; A61K 45/06; A61K 47/10; A61K 47/14; A61K 47/18; A61K 47/186; A61K 47/22; A61K 47/26; A61K 47/32; A61K 47/44

(57) ABSTRACT

Provided herein are gel, ointment, lotion, or cream formulations including an active pharmaceutical ingredient that includes a tropanyl moiety. The gel, lotion or cream formulations may include at least one low molecular weight (LMW) compound or a pharmaceutically acceptable salt thereof. Also provided herein are gel, ointment, lotion, or cream formulations including an active pharmaceutical agent such as atropine, pilocarpine, physostigmine, or travoprost.

### CROSS-REFERENCE TO RELATED APPLICATIONS

This application claims the benefit of U.S. Provisional Application No. 63/452,633 filed Mar. 16, 2023, U.S. Provisional Application No. 63/605,955 filed Dec. 4, 2023, and U.S. Provisional Application No. 63/549,789 filed Feb. 5, 2024, which are incorporated herein by reference in their entirety.

Tropane alkaloids include a class of bicyclic [3.2.1] alkaloids and secondary metabolites that contain a tropane ring in their chemical structure. Many tropane alkaloids occur naturally. Tropane alkaloids include cocaine, scopolamine, and atropine, among many others. Desirable pharmacological properties may be imparted through anticholinergic or stimulant activity of tropane alkaloids.

Therapeutic uses of tropane alkaloids are varied, and include anesthetic, anti-arrhythmic, anti-emetic, anti-histamine, anti-Parkinson, anti-spasmodic, bronchodilator, gastrointestinal, and ophthalmic uses. For example, Atropine is a muscarinic receptor antagonist, a type of anticholinergic agent, which blocks activity of the muscarinic acetylcholine receptor. By blocking the neurotransmitter acetylcholine at synapses in the central and peripheral nervous system, atropine has found use in treatment of poisoning by nerve agents and organophosphate insecticides, which destroy acetylcholinesterase by phosphorylation and trigger detrimental buildup of acetylcholine in the body. Atropine has also found use in treatment of amblyopia, hyperopia, or myopia due to its cycloplegic and mydriatic effects.

One challenge of tropane alkaloid therapeutics, including atropine therapeutics, is their chemical stability, including, for example, hydrolytic degradation. For example, atropine displays instability under basic, acidic, and oxidative conditions. Atropine degrades via hydrolysis to tropine and tropic acid, which degradation is accelerated under strongly acidic or basic conditions—atropine's hydrolytic degradation rate is least in the pH range of about 3-4. Furthermore, on loss of a water molecule, atropine converts to apoatropine, which itself degrades via hydrolysis to tropine and atropic acid.

Thus, with respect to therapeutic uses of tropane alkaloids, stable formulations are needed that address both formulation physical stability and the chemical instability of tropane alkaloids in formulations.

Travoprost is a prostaglandin is used for treating glaucoma or open-angle ocular hypertension.

Demodex-induced blepharitis is inflammation of the eyelids and eyelashes. It normally occurs when tiny oil/meibomian glands near the base of the eyelashes become clogged which causes eyelid inflammation, redness and irritation. Demodex mites are often the root cause of Demodex blepharitis and others associated with meibomian gland dysfunction and related ophthalmic diseases. Blepharitis commonly affects the eyes along the margins of the eyelids and eye lashes. Existing FDA-approved treatments for Demodex blepharitis include eye drops comprising an anti-parasitic. However, the existing treatment causes ocular irritation, stinging, burning sensation, contact dermatitis and/or allergy, and has a low rate of effectiveness for the active pharmaceutical ingredient which is used in killing Demodex mites. Thus, there is a need for improved treatments for Demodex blepharitis.

### BRIEF DESCRIPTION OF THE DRAWINGS

FIG. 1 shows the chemical stability of atropine in cream formulation 1 over time following exposure of the cream to 25 kGy eBeam sterilization and 40° C. at 75% relative humidity. Dashed lines represent 95% prediction interval.

FIG. 2 shows the total impurities from atropine in cream formulation 1 over time following exposure of the cream to 25 kGy eBeam sterilization and 40° C. at 75% relative humidity. Dashed lines represent 95% prediction interval.

FIG. 3 shows the chemical stability of atropine in cream formulation 2 over time following exposure of the cream to 25 kGy eBeam sterilization and 40° C. at 75% relative humidity. Dashed lines represent 95% prediction interval.

FIG. 4 shows the total impurities from atropine in cream formulation 2 over time following exposure of the cream to 25 kGy eBeam sterilization and 40° C. at 75% relative humidity. Dashed lines represent 95% prediction interval.

FIG. 5 shows the average atropine concentration in the aqueous humor target tissue following single dosing (drops to the eye or cream to the eye lid) of atropine on an equal molar basis with formulations comprising 0.05% w/w atropine (eye drop solution), 0.15% w/w atropine (cream), or 1% w/w atropine (cream).

FIG. 6 shows the same data in FIG. 5 plotted on a log scale.

FIG. 7 shows maximum pupil size diameter over time with 0.15% atropine cream (formulation 1 in Table 1) and eye drops.

FIG. 8 shows average systemic concentration over time with 0.05% atropine topical ophthalmic solution and cream (formulation 2 in Table 1) plotted on a log scale.

FIG. 9 shows the chemical stability of atropine in cream formulation 23 over time following exposure of the cream to 25 kGy eBeam sterilization and 40° C. at 75% relative humidity. Dashed lines represent 95% prediction interval.

FIG. 10 shows the travoprost free acid concentration in aqueous humor in a 14-day pharmacokinetics (PK) study in Yucatan minipigs using cream formulations comprising 0.004% to 0.0012% travoprost for treatment of glaucoma.

FIG. 11A shows tear break up time for a pilocarpine cream formulation comprising about 4% to about 8% w/w pilocarpine, compared to placebo, in a human trial for dry eye disease.

FIG. 11B shows vision improvement for a pilocarpine cream formulation comprising about 4% to about 8% w/w pilocarpine, compared to placebo, in a human trial for dry eye disease.

FIG. 12 shows lethality towards mites that cause Demodex blepharitis using a 1% physostigmine cream formulation versus a lotilaner 0.25% eye drop formulation.

FIG. 13 shows the atropine concentration in aqueous humor in a 14 day repeat dose pharmacokinetics (PK) study in Yucatan minipigs using 0.05% ophthalmic solution, and cream formulations comprising 0.15%, and 1% atropine for treatment of progressive myopia.

FIG. 14 shows data from drug-excipient compatibility testing between travoprost and functional excipients.

FIG. 15 shows in vitro active pharmaceutical ingredient (API) release profiles for 0.5% w/w, 1% w/w, and 2% w/w physostigmine cream formulations.

FIG. 16A shows in vitro penetration across a synthetic skin membrane for atropine cream formulations comprising about 0.05% w/w to about 0.25% w/w atropine and about 0.05% w/w to about 5% w/w carbomer.

FIG. 16B shows the experimental set up for in vitro penetration testing.

FIG. 17 shows certain degradation pathways for physostigmine.

FIG. 18 illustrates the formulation viscosity relationship of carbomer and SEPINEO® P600.

FIG. 19 illustrates the relationship between % assay of physostigmine and formulation pH.

FIG. 20 shows a flowchart for preparation of physostigmine formulations.

FIG. 21 shows chemical stability trends for certain formulations of Example 13, namely PHY012 and PHY052 at 40° C.

FIG. 22 shows total impurity profiles for certain formulations of Example 13, namely PHY012 and PHY052 at 40° C.

FIG. 23 shows physical stability (pH) profile of formulation PHY052 of Example 13 at 40° C./75% relative humidity (RH).

FIG. 24 shows physical stability (viscosity) of formulation PHY052 of Example 13 at 40° C./75% RH

FIG. 25A shows stability studies and trend analysis for formulation screening and determination of product shelf-life.

FIG. 25B shows aesthetic performance prediction and tube filling parameters.

### DETAILED DESCRIPTION

Topical application of active pharmaceutical ingredients may be via one of many composition types, including liquid, ointment, gel, lotion, and cream formats. Each topical application format having its own advantages and disadvantages. For example, liquids are often absorbed rapidly on application but are limited to topical mucosal membrane applications where a pH misalignment with that of the mucosal membrane for the sake of chemical stability may cause discomfort on application—applying an acidic solution to a mucosal membrane may cause a burning or stinging sensation. Creams are generally water rich environments (especially for oil-in-water emulsion cream systems) and subject active pharmaceutical ingredients therein to hydrolytic or pH sensitive conditions. Ointments, being oily and generally water depleted, provide a protective barrier to the skin but are not absorbed themselves, which leaves a greasy residue on the target location. Importantly, topical formulations must also meet certain regulations regarding resistance to antimicrobial or antibacterial growth.

Stable cream formulations including tropane alkaloids have been discovered. Notwithstanding the aqueous nature of the cream formulations and tropane alkaloids' susceptibility to hydrolytic degradation, the tropane alkaloids present in the creams are chemically stable for commercially relevant time periods. It has also been discovered that including at least one polycyclic nucleobase in the creams improves stability of the cream formulations. It has further been discovered that including low molecular weight compounds improves stability of the cream formulations. Without being bound by theory, atropine may be intercalating with the stabilizers and forming steric hindrance around atropine, and the steric hindrance protects atropine from degradation to tropic acid. In addition to forming steric hindrance around the atropine molecule, the stabilizers may effectively protect the atropine molecule from E-beam irradiation. A further benefit of the tropanyl creams provided herein is that they do not require common preservatives such as benzalkonium chloride or parabens in order to remain usable during commercially relevant storage periods. Additionally, the cream formulations have been found to impart improved pharmacokinetic profiles over those from an alternative topical application format such aseye drops.

Additional cream formulations are provided comprising atropine, pilocarpine, travopost, or physostigmine. It has been discovered that inclusion of one or more preservatives, and at least one antioxidant that partitions into the appropriate phase (aqueous phase or oil phase) of the cream formulation and at least one antioxidant that partitions into the other phase (oil phase or aqueous phase) of the cream formulation, improves stability of the cream formulation. Further, mixing in an active agent such as physostigmine at the final step during manufacturing after pre-adjusting pH, as shown in FIG. 20, also increases stability of an active agent (e.g., physostigmine) in the cream formulation.

The addition process of adding antioxidant(s) and/or preservative(s) depends on the solubility in the respective solvent phase (either oil or aqueous phase) and the API solubility/stability. Co-mixing or co-dissolving of the antioxidant(s) with the API(s) in the formulation compounding process can protect API(s) from degradation during the manufacturing processes in addition to the enhanced stability of the finished drug product.

Physostigmine is a reversible anticholinesterase which effectively increases the concentration of acetylcholine at the sites of cholinergic neurotransmission. The action of acetylcholine is normally very transient because of its hydrolysis by the enzyme, acetylcholinesterase. Physostigmine inhibits the destructive action of acetylcholinesterase and thereby prolongs and exaggerates the effect of acetylcholine. Acetylcholinesterase inhibitors have been widely used as pesticides to kill arthropods such as the Demodex mites by potentiating cholinergic neurotransmission, resulting in the continual over-excitation of nerve-to-nerve and nerve-to-muscle communication, and eventual paralysis and death. The levorotary enantiomer (negative optical isomer) of physostigmine has much higher efficacy (˜100× to 1000×) in mite killing than that of its positive counterpart. Advantageously, physostigmine in topical cream dosage form can treat Demodex blepharitis by directly contacting the mites at the target sites for a sustained duration compared to eye drops. Accordingly, provided herein are physostigmine salicylate based topical creams with minimal degradation of physostigmine. The physostigmine-based cream formulations are physically and chemically stable for over 4 months under accelerated stability conditions (e.g., 40° C. and 75% relative humidity (RH)).

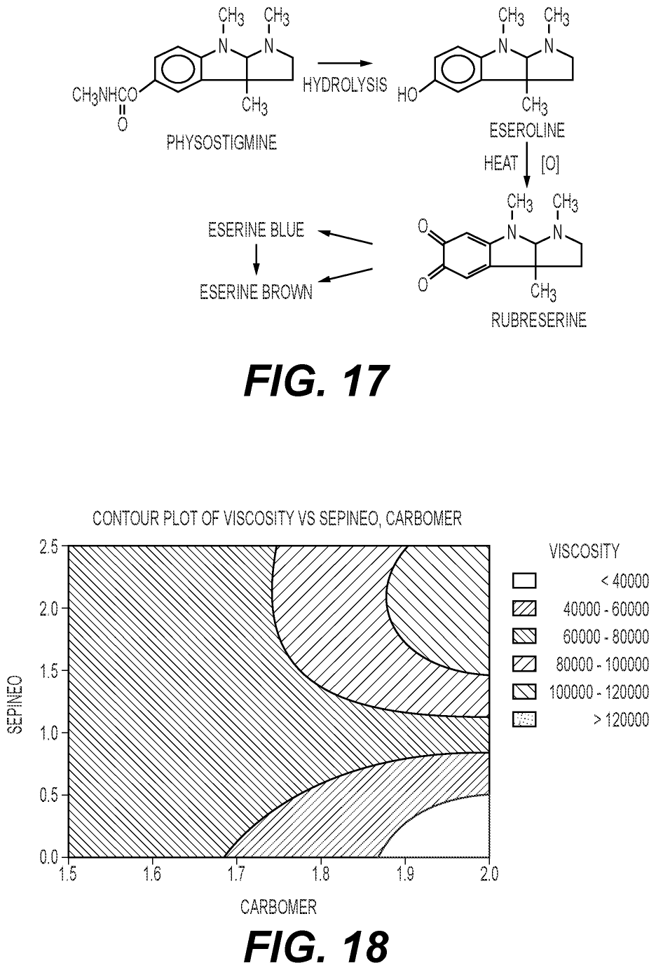

Physostigmine salicylate undergoes rapid degradation and oxidation in aqueous medium. The degradation pathways of physostigmine salicylate in aqueous medium are shown in FIG. 17. Described herein are compositions and methods/processes of making physostigmine salicylate based topical creams with minimal degradation of physostigmine. The physostigmine-based cream formulations described herein are physically and/or chemically stable for over 4 months under accelerated stability conditions (e.g., 40° C. and 75% RH).

### Definitions

While the disclosure has been described in detail and with reference to specific embodiments thereof, it will be apparent to one skilled in the art that various changes and modifications can be made without departing from the spirit and scope of the disclosure.

Groupings of alternative elements or embodiments disclosed herein may be referred to and claimed individually or in any combination with other members of the group or other elements found herein.

Unless otherwise defined, all technical and scientific terms used herein are accorded the meaning commonly known to one with ordinary skill in the art.

Those skilled in the art will recognize, or be able to ascertain, using no more than routine experimentation, many equivalents of the specific embodiments of the invention described herein. Such equivalents are intended to be encompassed by the claims below.

Certain terms, whether used alone or as part of a phrase or another term, are defined below.

The articles “a” and “an” refer to one or to more than one of the grammatical object of the article.

Numerical values relating to measurements are subject to measurement errors that place limits on their accuracy. For this reason, all numerical values provided herein, unless otherwise indicated, are to be understood as being modified by the term “about.” Accordingly, the term about refers to an error of ±10%. Otherwise, the last decimal place of a numerical value provided herein indicates its degree of accuracy. Where no other error margins are given, the maximum margin is ascertained by applying the rounding-off convention to the last decimal place or last significant digit when a decimal is not present in the given numerical value.

The term “alkanalyl” refers to branched, cyclic, or straight chain, or a combination thereof, saturated hydrocarbon moieties that include at least one aldehyde.

The term “alkanonyl” refers to branched, cyclic, or straight chain, or a combination thereof, saturated hydrocarbon moieties that include at least one ketone.

The term “alkenalyl” refers to branched, cyclic, or straight chain, or a combination thereof, hydrocarbon moieties that include at least one aldehyde and at least one carbon-carbon double bond.

The term “alkanolyl” refers to branched, cyclic, or straight chain, or a combination thereof, saturated hydrocarbon moieties that include at least one hydroxyl.

The term “alkenonyl” refers to branched, cyclic, or straight chain, or a combination thereof, hydrocarbon moieties that include at least one ketone and at least one carbon-carbon double bond.

The term “alkenyl” refers to branched, cyclic, or straight chain, or a combination thereof, hydrocarbon moieties that include at least one carbon-carbon double bond.

The term “alkyl” refers to branched, cyclic, or straight chain, or a combination thereof, saturated hydrocarbon moieties.

The term “halo” refers to a halogen.

The term “amelioration” means a lessening of severity of at least one indicator of a condition or disease, such as a delay or slowing in the progression of one or more indicators of a condition or disease. The severity of indicators may be determined by subjective or objective measures which are known to those skilled in the art.

The term “Cₑₖ” refers to a moiety comprising e to k carbon atoms, wherein e and k are integers.

The terms “effective amount” and “therapeutically effective amount” refer to an amount of active ingredient, such as a compound described herein, administered to a subject, either as a single dose or as part of a series of doses, which produces a desired effect. In general, the effective amount can be estimated initially either in cell culture assays or in mammalian animal models, for example, in non-human primates, mice, rabbits, dogs, or pigs. The animal model may also be used to determine the appropriate concentration range and route of administration. Such information can then be used to determine useful doses and routes for administration in non-human subjects and human subjects.

The terms “cream” and “pharmaceutical cream” refer to a mixture of at least one active pharmaceutical ingredient described herein with a pharmaceutically acceptable carrier. Pharmaceutical creams facilitate administration of at least one active pharmaceutical ingredient to a patient or subject.

The term “pharmaceutically acceptable carrier” means a pharmaceutically acceptable material, composition, or carrier, such as a liquid filler, solid filler, stabilizer, dispersing agent, suspending agent, diluent, excipient, thickening agent, solvent, or encapsulating material, involved in carrying or transporting at least one active pharmaceutical ingredient described herein within or to the patient such that the active pharmaceutical ingredient may perform its intended function. A given carrier must be “acceptable” in the sense of being compatible with the other ingredients of a particular cream formulation, including the active pharmaceutical ingredients described herein, and not injurious to the patient. Other ingredients that may be included in the pharmaceutical creams described herein are known in the art and described, for example, in “Remington's Pharmaceutical Sciences” (Genaro (Ed.), Mack Publishing Co., 1985), the entire content of which is incorporated herein by reference.

The term “pharmaceutically acceptable salt” refers to derivatives of the disclosed compounds wherein the parent compound is modified by converting an existing acid or base moiety to its salt form. Pharmaceutically acceptable salts can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two solvents. Lists of suitable salts are found in “Handbook of Pharmaceutical Salts: Properties, Selection, and Use” (P. Henrich Stahl & Camille G. Wermuth (Eds.), VHCA & Wiley-VCH, 2002), the entire content of which is incorporated herein by reference. Reference to a type or class of compound herein may be understood as including pharmaceutically acceptable salts of that compound.

The term “refractory disease” refers to a disease that continues to progress during treatment with a pharmaceutical ingredient other than the active pharmaceutical ingredients provided herein, partially responds to the other treatment, or transiently responds to the other treatment. The term may be applied to each of the diseases referred to herein.

The terms “treatment” or “treating” refer to the application of one or more specific procedures used for the amelioration of a disease. A “prophylactic” treatment, refers to reducing the rate of progression of the disease or condition being treated, delaying the onset of that disease or condition, or reducing the severity of its onset.

Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the described subject matter and does not pose a limitation on the scope of the subject matter otherwise claimed. No language in the specification should be construed as indicating any non-claimed element essential to practicing the described subject matter.

Groupings of alternative elements or embodiments of this disclosure are not to be construed as limitations. Each group member may be referred to and claimed individually or in any combination with other members of the group or other elements found herein. Furthermore, a recited member of a group may be included in, or excluded from, another recited group for reasons of convenience or patentability.

Reference, if any, made to a patent document or other publication in this specification serves as an incorporation herein by reference of the entire content of such document or publication.

Embodiments of this disclosure are illustrative. Accordingly, the present disclosure is not limited to that precisely as shown and described.

### Compositions

In some embodiments, provided herein are certain compositions, which may be pharmaceutical compositions suitable for therapeutic use. The compositions may include one or more pharmaceutically acceptable carriers.

Thus, in some embodiments, provided herein are compositions comprising: one or more purified tropane alkaloid or a pharmaceutically acceptable salt thereof; and one or more purified polycyclic nucleobase or a pharmaceutically acceptable salt thereof. Such tropane alkaloids and polycyclic nucleobases are described further below. In some embodiments, the composition comprises two different purified tropane alkaloids or pharmaceutically acceptable salts thereof, and one, two, or three purified polycyclic nucleobases or pharmaceutically acceptable salts thereof. In some embodiments, the pharmaceutical compositions may be provided as a pharmaceutical cream.

In some embodiments, provided herein is a topical formulation comprising an active pharmaceutical ingredient comprising a tropanyl group. In some embodiments, the topical formulation is an ophthalmic formulation. In some embodiments, the topical formulation is a gel, a lotion, or a cream.

In some embodiments, the topical formulation has a viscosity ranging from about 2000 to about 200000 centipoise (cP). In some embodiments, the topical formulation has a viscosity ranging from about 2000 to about 70000 centipoise (cP). In some embodiments, the topical formulation has a viscosity ranging from about 2000 to about 50000 centipoise (cP). In some embodiments, the topical formulation has a viscosity ranging from about 10000 to about 75000 centipoise (cP). In some embodiments, the topical formulation has a viscosity ranging from about 10000 to about 50000 centipoise (cP). In some embodiments, the topical formulation has a viscosity ranging from about 10000 to about 25000 centipoise (cP). In some embodiments, the topical formulation has a viscosity ranging from about 25000 to about 200000 centipoise (cP). In some embodiments, the topical formulation has a viscosity ranging from about 25000 to about 75000 centipoise (cP). In some embodiments, the topical formulation is an oil-in-water (o/w) formulation.

### Creams, Gels, and Lotions

Formulations have been discovered for mitigation of the hydrolysis of tropane alkaloids to their corresponding hydrolysis products, including tropine or the like. Accordingly, formulations, such as cream, gel, or lotion formulations, have been discovered that remain physically and chemically stable on continued exposure to storage conditions relevant to commercial manufacture, distribution, and consumer usage. The formulations are useful in enabling and promoting penetration of tropane alkaloids from the cream through the skin of a subject or patient, and subsequent delivery of the tropane alkaloids to target tissue underlying the skin. In some embodiments, the target tissue may include a portion of the subject's skin, or blood vessels, or ultimately systemic distribution through the vasculature. In some embodiments, the target tissue may be an eye or related ocular anatomy, including topical application thereto.

Thus, in some embodiments, provided herein are gel formulations comprising an active pharmaceutical ingredient that includes a tropanyl moiety. In some embodiments, provided herein are lotion formulations comprising an active pharmaceutical ingredient that includes a tropanyl moiety. In some embodiments, provided herein are cream formulations comprising an active pharmaceutical ingredient that includes a tropanyl moiety. In some embodiments, the creams are oil-in-water (o/w) emulsion creams wherein the creams include at least 50 or at least 60% w/w water. In some embodiments, provided herein are cream formulations comprising an active pharmaceutical ingredient that includes a tropane alkaloid. In some embodiments, the tropane alkaloid is contained within the oil phase of the oil-in-water emulsion, which, without being bound by theory, may mitigate hydrolysis or chemical degradation of the tropane alkaloid. In some embodiments, provided herein are cream formulations comprising an active pharmaceutical ingredient that includes a tropinyl moiety. In some embodiments, the active pharmaceutical ingredient that includes a tropanyl moiety, a tropinyl moiety, or a tropane alkaloid is an anticholinergic compound. Anticholinergic and cholinergic agents generally have opposite effects in a subject since the former blocks the action of acetylcholine and the latter mimics the action of acetylcholine. For example, anticholinergic agents may inhibit salivation or sweating while cholinergic agents may promote salivation or sweating. In some embodiments, the active pharmaceutical ingredient is atropine, benzatropine, cocaine, homatropine, hyoscyamine, scopolamine, tiotropium, tropisetron, or trospium, or a pharmaceutically acceptable salt thereof.

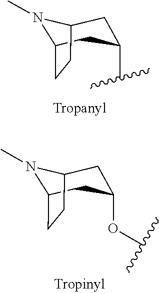

In some embodiments, provided herein are cream formulations comprising an active pharmaceutical ingredient that is atropine. In some embodiments, provided herein are gel formulations comprising an active pharmaceutical ingredient that is atropine. In some embodiments, provided herein are lotion formulations comprising an active pharmaceutical ingredient that is atropine.

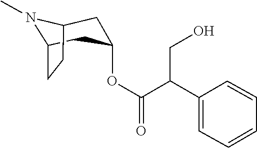

### (1R,3r,5S)-8-methyl-8-azabicyclo[3.2.1 ]octan-3-yl 3-hydroxy-2-phenylpropanoate (Atropine)

In some embodiments, the gel, ointment, lotion, or cream formulations comprise atropine, wherein the atropine comprises more than 50% by weight of (1R,3r,5S)-8-methyl-8-azabicyclo[3.2.1]octan-3-yl (S)-3-hydroxy-2-phenylpropanoate. In some embodiments, the gel, ointment, lotion, or cream formulations comprise atropine, wherein the atropine comprises more than 50% by weight of (1R,3r,5S)-8-methyl-8-azabicyclo[3.2.1]octan-3-yl (R)-3-hydroxy-2-phenylpropanoate. In some embodiments, the gel, ointment, lotion, or cream formulations comprise atropine, wherein the atropine comprises less than 10% by weight of (1R,3r,5S)-8-methyl-8-azabicyclo[3.2.1 ]octan-3-yl (S)-3-hydroxy-2-phenylpropanoate. In some embodiments, the gel, ointment, lotion, or cream formulations comprise atropine, wherein the atropine comprises less than 10% by weight of (1R,3r,5S)-8-methyl-8-azabicyclo[3.2.1 ]octan-3-yl (R)-3-hydroxy-2-phenylpropanoate.

In some embodiments, the gel, ointment, lotion, or cream formulations further comprise caffeine or derivatives thereof. In some of such embodiments, the caffeine or derivatives thereof can serve as chemical stabilizing agents and/or as emollients.

In some embodiments, the gel, ointment, lotion, or cream formulations further include a polycyclic nucleobase or a pharmaceutically acceptable salt thereof. In some embodiments, the nucleobase is a bicyclic-or tricyclic nucleobase. In some embodiments, the gel, ointment, lotion, or cream formulations further include a heterocyclic compound having 9, or more, ring atoms. In some embodiments, the 9 ring atoms include at least 5 carbon atoms. In some embodiments, the 9 ring atoms include at least 3 nitrogen atoms. In some embodiments, the 9 ring atoms include 4 nitrogen atoms. In some embodiments, the heterocyclic compound is an aromatic heterocyclic compound that includes a fused six- and five-membered ring system. In some embodiments, the heterocyclic compound is a purine compound. In some embodiments, the heterocyclic compound is an oxo-purine (e.g., an 8-oxo-purine) compound. In some embodiments, the heterocyclic compound is a halo-purine compound. In some embodiments, the heterocyclic compound is a thio-purine compound. In some embodiments, the heterocyclic compound is a methylpurine, or an 8-oxo-methylpurine. In some embodiments, the heterocyclic compound is a monomethylpurine, an 8-oxo-monomethylpurine, a dimethylpurine, an 8-oxo-dimethylpurine, a trimethylpurine, or an 8-oxo-trimethylpurine compound. In some embodiments, the purine compound is azathioprine, adenine, 2-aminopurine, 2,6-diaminopurine, 2,6,8-triaminopurine, 2-amino-6-methoxy-purine, 2-chloro-6-aminopurine, 2-fluoro-6-aminopurine, guanine, thioguanine, isoguanine, purine, mercaptopurine, hypoxanthine, xanthine, 1-methylxanthine, 3-methylxanthine, 7-methylxanthine, 1,3-dimethylxanthine (theophylline), 3,7-dimethylxanthine (theobromine), 1,7-dimethylxanthine (paraxanthine), 1,3,7-trimethylxanthine (caffeine), 8-chloro-1,3-dimethylxanthine, uric acid, 1-methyluric acid, 3-methyluric acid, 7-methyluric acid, 1,3-dimethyluric acid, 3,7-dimethyluric acid, 1,7-dimethyluric acid, and 1,3,7-trimethyluric acid.

In some embodiments, the heterocyclic compound has the following formula:

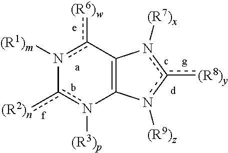

or a pharmaceutically acceptable salt thereof,
wherein
each of [symbol] a, [symbol] b, [symbol] c, [symbol] d, [symbol] e, [symbol] f, and [symbol] g is, independently, a single bond or a double bond;
each of m, n, p, w, x, y, and z is, independently, 0 or 1;
each of R¹, R³, R⁷, and R⁹ is, independently, H, C₁₋₆ alkyl, C₁₋₆ alkanolyl, C₁₋₆ haloalkyl, C₂₋₆ alkenyl, C₂₋₈ alkanalyl, C₃₋₈ alkenalyl, C₂₋₈ alkanonyl, or C₄₋₈ alkenonyl;
each of R², R⁶, and R⁸ is, independently, H, halo, O—(C₁₋₃ alkyl), NH₂, N(H)(C₁₋₃ alkyl), N(C₁₋₃ alkyl)(C₁₋₃ alkyl), S, or O.

In some embodiments: each of R¹, R³, R⁷, and R⁹ is, independently, H or C₁₋₃ alkyl; and each of R², R⁶, and R⁸ is R² is, independently, H, F, Cl, NH₂, OCH₃, N(H) (C₁₋₃ alkyl), N(C₁₋₃ alkyl)(C₁₋₃ alkyl), S, or O. In some embodiments of the heterocyclic compound formula, each alkyl is, independently, methyl, ethyl, propyl, or isopropyl.

In some embodiments, halo refers to F, Cl, or I. In some embodiments, the haloalkyl moiety may be a mono-, di-, tri-, tetra-, penta-, or hexa-haloalkyl wherein each halogen is independently selected from F or Cl. In some embodiments, the haloalkyl includes a CFH₂, CF₂H, CF₃, C₂FH₄, C₂F₂H₃, C₂F₃H₂, C₂F₄H, or CF₅ moiety, or a combination thereof.

In some embodiments of the heterocyclic compound formula, each of R¹, R³, R⁷, and R⁹ is, independently, H or methyl. In some embodiments, when R¹ is H or C₁₋₃ alkyl (e.g., methyl), [symbol] a is a single bond. In some embodiments, when R³ is H or C₁₋₃ alkyl (e.g., methyl), [symbol] b is a single bond. In some embodiments, when R⁷ is H or C₁₋₃ alkyl (e.g., methyl), [symbol] c is a single bond. In some embodiments, when R⁹ is H or C₁₋₃ alkyl (e.g., methyl), [symbol] d is a single bond.

In some embodiments, when m is 0, [symbol] a is a double bond. In some embodiments, when p is 0, [symbol] b is a double bond. In some embodiments, when x is 0, [symbol] c is a double bond. In some embodiments, when z is 0, [symbol] d is a double bond.

In some embodiments, each of R², R⁶, and R⁸ is, independently, H, NH₂, S, or O. In some embodiments, when R² is S or O, [symbol] f is a double bond and [symbol] b is a single bond. In some embodiments, when R⁶ is S or O, [symbol] e is a double bond and [symbol] a is a single bond. In some embodiments, when R⁸ is S or O, [symbol] g is a double bond, [symbol] c is a single bond, and [symbol] d is a single bond.

In some embodiments, the gel, ointment, lotion, or cream formulations herein are physically and chemically stable, which stability persists for commercially relevant time periods, including at least 3 months, up to 6 months, or longer. Example 2 describes such stability.

In some embodiments, a low molecular weight (LMW) compound with one or more aromatic rings or compounds with conjugated structures may be included in the formulations. These LMW compounds, which may include histidine, sorbic acid (conjugated molecule), benzoic acid, 2,5-dihydroxybenzoic acid, 2,5-dihydroxyterephthalic acid, 1,4-dihydroxy-2-naphthoic acid, 3,7-dihydroxy-2-naphthoic acid, their derivatives, and their pharmaceutically acceptable salt forms (i.e., sodium, potassium, ammonium, chloride, sulfate, magnesium, and/or calcium), may be used as stabilizers to stabilize (or contribute to the stabilization of) active pharmaceutical ingredients (APIs) in cream formulations. The chemical structures of histidine and sorbic acid are shown below. In some embodiments, the pKa of an atropine stabilizer is similar to the desired pH of the gel, ointment, lotion, or cream formulation comprising atropine (e.g., a pKa of about 3 to about 6). By way of example only, without limitation, the pKa of sorbic acid is about 4.76 and the pH of a gel, ointment, lotion, or cream formulation may be about 4.80. In some embodiments, the sorbic acid may be a free acid. In some embodiments, the sorbic acid may be a salt. In some embodiments, sorbic acid stabilizes atropine in a cream formulation as shown in Table 8 herein; similarly so for a gel or lotion formulation. Advantageously, sorbic acid and salts thereof are a cheaper alternative, and thereby commercially beneficial, to other stabilizers such as histidine.

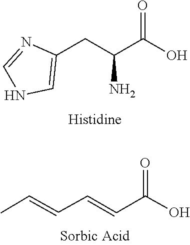

In some embodiments, some LMW compounds such as sinapinic acid, α-cyano-4-hydroxycinnamic acid (HCCA), 4-chloro-α-cyanocinnamic acid, and their pharmaceutically acceptable salt forms (i.e., sodium potassium, ammonium, magnesium, and/or calcium) may be used as chemical stabilizers to stabilizer APIs in gel, ointment, lotion, or cream formulations. The chemical structure of HCCA is shown below.

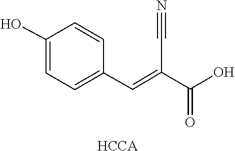 [SMILES: N#C/C(=C\c1ccc(O)cc1)C(=O)O ; "HCCA"]

In some embodiments, the stabilizing agents may be LMW compounds that have one or more aromatic ring structures without a carboxylic acid functionality. These compounds may be used as chemical stabilizers to stabilizer APIs in cream formulations. The chemical structure of one such compound is dithranol is shown below.

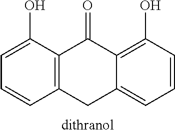 [SMILES: O=C1c2c(O)cccc2Cc2cccc(O)c21]

In some embodiments, the LMW compound has a molecular weight of about 50 g/mol to about 1000 g/mol, about 75 g/mol to about 900 g/mol, about 100 g/mol to about 800 g/mol, about 125 g/mol to about 700 g/mol, about 150 g/mol to about 600 g/mol, about 175 g/mol to about 500 g/mol, about 200 g/mol to about 400 g/mol, or about 225 g/mol to about 300 g/mol.

In some embodiments, the gel, ointment, lotion, or cream formulation may further include at least one low molecular weight compound or a pharmaceutically acceptable salt thereof. The at least one low molecular weight compound may include one or more aromatic ring structures. The LMW compound may be present as a salt including salts selected from sodium, potassium, ammonium, magnesium, calcium salts, or a combination thereof.

In some embodiments, at least one low molecular weight compound or its pharmaceutically acceptable salt thereof stabilizes active pharmaceutical ingredients (APIs) in the cream formulation.

In some embodiments, at least one low molecular weight compound includes caffeine, benzoic acid, sinapinic acid, α-cyano-4-hydroxycinnamic acid, histidine, or dithranol, or a pharmaceutically acceptable salt thereof, or a combination thereof. In some embodiments, the at least one low molecular weight compound includes benzoic acid, sinapinic acid, α-cyano-4-hydroxycinnamic acid, histidine, or dithranol, or a pharmaceutically acceptable salt thereof, or a combination thereof. In some embodiments, the at least one low molecular weight compound is caffeine. In some embodiments, the at least one low molecular weight compound is histidine. In some embodiments, the at least one low molecular weight compound is α-cyano-4-hydroxycinnamic acid.

In some embodiments of the gel, ointment, lotion, or cream formulation, the at least one low molecular weight compound is present in the gel, ointment, lotion, or cream formulation in an amount of about 0.01% to about 1% w/w.

In some embodiments of the gel, ointment, lotion, or cream formulation, the at least one low molecular weight compound is present in the gel, ointment, lotion, or cream formulation in an amount of about 0.05% to about 0.7% w/w.

In some embodiments, the gel, ointment, lotion, or cream formulations include about 0.01 to about 2.5% w/w tropane alkaloid or a pharmaceutically acceptable salt thereof. In some embodiments, the gel, ointment, lotion, or cream formulations include about 0.05 to about 2.5% w/w tropane alkaloid or a pharmaceutically acceptable salt thereof. In some embodiments, the gel, ointment, lotion, or cream formulations include about 0.01, 0.05, 0.1, 0.15, 0.2, 0.3, 0.4, 0.5, 0.6, 0.7, 0.8, 0.9, 1, 1.1, 1.2, 1.5, or 2.5% w/w, or any range there between, of one or more tropane alkaloid or a pharmaceutically acceptable salt thereof. In some embodiments, the one or more tropane alkaloid or pharmaceutically acceptable salt thereof is, independently, atropine, benzatropine, cocaine, homatropine, hyoscyamine, scopolamine, tiotropium, tropisetron, or trospium, or a pharmaceutically acceptable salt thereof. In some embodiments, the tropane alkaloid or pharmaceutically acceptable salt thereof is an atropine sulfate. In some embodiments, the gels, lotions, or creams herein include about 0.01, 0.05, 0.08, 0.15, 0.5, or 1% w/w of an atropine sulfate. As used herein the % w/w of atropine, regardless of any salt form used in the gel, ointment, lotion, or cream formulation, is calculated based on the free base.

In some embodiments, the gel, ointment, lotion, or cream formulations herein may include about 0.01 to about 0.3% w/w of a polycyclic nucleobase or a pharmaceutically acceptable salt thereof. In some embodiments, the gel, ointment, lotion, or cream formulations herein may include about 0.05 to about 0.3% w/w of a polycyclic nucleobase or a pharmaceutically acceptable salt thereof. In some embodiments, the gels, lotions, or creams herein may include about 0.1 to about 0.2% w/w of a polycyclic nucleobase or a pharmaceutically acceptable salt thereof. In some embodiments, the gels, lotions, or creams include about 0.16% w/w of 1,3,7-trimethylxanthine. In some embodiments, the gel, ointment, lotion, or cream formulations are polycyclic nucleobase-free (e.g., the creams do not include a polycyclic nucleobase or a pharmaceutically acceptable salt thereof, e.g., the gels, lotions, or creams do not include 1,3,7-trimethylxanthine).

In some embodiments, the gel, ointment, lotion, or cream formulation comprises about 0.0005 to about 0.001% w/w of polyquaternium-1 or a pharmaceutically acceptable salt thereof, and about 0.00025 to about 0.0005% w/w of alexidine or a pharmaceutically acceptable salt thereof, wherein the gel, ointment, lotion, or cream formulation is otherwise preservative-free, and wherein the gel, ointment, lotion, or cream formulation optionally includes an active pharmaceutical ingredient.

In some embodiments, the gel, ointment, lotion, or cream formulation, comprises a stabilizer selected from histidine, sorbic acid, or alpha cinnamic acid, or a pharmaceutically acceptable salt thereof, and comprises about 0.0005 to about 0.001% w/w of polyquaternium-1 or a pharmaceutically acceptable salt thereof, and about 0.00025 to about 0.0005% w/w of alexidine or a pharmaceutically acceptable salt thereof, wherein the gel, ointment, lotion, or cream formulation is otherwise preservative-free, and wherein the cream formulation optionally includes an active pharmaceutical ingredient.

In some embodiments, the gels, lotions, or creams provided herein include less than about 0.25% w/w of a preservative. In some embodiments, the creams provided herein include not more than about 0.002% w/w of one or more preservatives. In some embodiments, the preservative includes parabens, but not benzalkonium chloride (BAK). In some embodiments, the creams provided herein do not include BAK (i.e., BAK-free) or parabens (e.g., methylparaben, ethylparaben, propylparaben, and butylparaben) (i.e., paraben-free). In some embodiments, the creams provided herein include about 0.001 to about 0.003% w/w preservative. In some embodiments, the gels, lotions, or creams provided herein include about 0.002% w/w preservative, wherein the preservative includes a biguanide compound (e.g., alexidine or a pharmaceutically acceptable salt thereof) and a polyquaternium compound (e.g., polyquaternium-1 or a pharmaceutically acceptable salt thereof (PQ-1)). In some embodiments, the gels, lotions, or creams provided herein include not more than about 0.0015% w/w of one or more preservatives. In some embodiments, the gels, lotions, or creams provided herein include about 0.001% w/w of PQ-1, and about 0.0005% w/w of alexidine or a pharmaceutically acceptable salt thereof, and zero % w/w parabens. In some embodiments, the gels, lotions, or creams provided herein include about 0.0005% w/w (e.g., about 5 PPM) of PQ-1, and about 0.00025% w/w (e.g., about 2.5 ppm) of alexidine or a pharmaceutically acceptable salt thereof, and zero % w/w parabens. In some embodiments, the gels, lotions, or creams include PQ-1 and alexidine or a pharmaceutically acceptable salt thereof in a ratio of about 2:1, respectively.

In some embodiments, the gels, lotions, or creams include a first pharmaceutically acceptable carrier, which is a copolymer of acrylamide and sodium acryloyldimethyl taurate, isohexadecane, and polysorbate 80, which are components of a product marketed as SEPINEO® P-600. In some embodiments, the first pharmaceutically acceptable carrier is present in about 0.5 to about 2% w/w, e.g., about 1% w/w. In some embodiments, the gels, lotions, or creams include a second pharmaceutically acceptable carrier, which is a polymer of acrylic acid cross-linked with one or more allyl ether of a polyalcohol, which is a component of a product marketed as carbomer homopolymer. In some embodiments, the second pharmaceutically acceptable carrier is present in about 1 to about 3% w/w, e.g., about 2% w/w.

As used herein, carbomer refers to polyacrylic acid polymers. Carbomer may also be referred to as CARBOPOL®. CARBOPOL® 980 refers to an excipient sold by, e.g., by Lubrizol, and has a Chemical Abstracts Service Registry Number (CAS RN) of 139637-85-7. CARBOPOL® 980 is described by Lubrizol as a homopolymer of acrylic acid crosslinked with allyl sucrose or allyl pentaerythritol, a cosolvent system made using ethyl acetate and cyclohexane, and having viscosity, cP (0.5 wt % at pH 7.5), of 40,000-60,000. Contemplated within the scope of embodiments presented herein are other homopolymers including, and not limited to, the CARBOPOL® polymers in the table below. Carbomer homopolymer type C refers to allyl pentaerythritol crosslinked polyacrylic acid.

| | Polymerization | Product | Viscosity, cP (0.5 |
|---|---|---|---|
| CARBOPOL ® | Solvent | Type | wt % at pH 7.5) |
| 71G NF | Ethyl Acetate | Homopolymer | 4,000-11,000 |
| 971P NF | Ethyl Acetate | Homopolymer | 4,000-11,000 |
| 974P NF | Ethyl Acetate | Homopolymer | 29,400-39,400 |
| 981 NF | Cosolvent¹ | Homopolymer | 4,000-10,000 |
| 5984 EP | Cosolvent¹ | Homopolymer | 30,500-39,400 |
| 934 NF | Benzene | Homopolymer | 30,500-39,400 |
| 934P NF | Benzene | Homopolymer | 29,400-39,400 |
| 940 NF | Benzene | Homopolymer | 40,000-60,000 |
| 941 NF | Benzene | Homopolymer | 4,000-10,000 |

¹Cosolvent system made using cyclohexane and ethyl acetate

As used herein SEPINEO® P600 refers to a copolymer sold by e.g., Seppic, and is associated with CAS RNs of 38193-60-1 (3 component acrylamide-sodium 2-acrylamido-2-methylpropanesulfonate copolymer)/93685-80-04 (isohexadecane)/9005-65-6 (polyoxyethylene sorbitan monooleate). SEPINEO® P600 is described by Seppic as a polymer of acrylamide/sodium acryloyldimethyl taurate copolymer/isohexadecane and polysorbate 80.

In some embodiments, the gels, lotions, or creams include a buffer having a pKₐ of between about 2.5 and 7. In some embodiments, the buffer includes at least one pKₐ of about 3, 4, 5, 6, or 7, or any range there between. In some embodiments, the buffer includes at least one pKₐ between about 3 and 6.5. In some embodiments, the buffer is a citrate or citric acid buffer. In some embodiments, the buffer concentration is present in the creams at about 0.05% to about 5% w/w. In some embodiments, the gel, ointment, lotion, or cream formulations include about 20% w/w to about 70% w/w of water. In some embodiments, the gels, lotions, or creams include about 0.01% w/w to about 0.5% w/w of sodium citrate, potassium citrate, magnesium citrate, or citric acid buffer. In some embodiments, the gels, lotions, or creams include about 0.05% w/w to about 0.5% w/w of a sodium citrate, potassium citrate, magnesium citrate, or citric acid buffer.

In some embodiments, the pH of the creams is about 4 to about 6, e.g., about 5, e.g., about 4.5, 4.6, 4.7, 4.8, or 4.9. In some embodiments, the pH of the gels, lotions, or creams is about 4.5 to about 5.

In some embodiments, the gels, lotions, or creams include a molar ratio of tropane alkaloid or pharmaceutically acceptable salt thereof to polycyclic nucleobase or pharmaceutically acceptable salt thereof of about (4-5):1, e.g., about 4.2:1.

Thus, in some embodiments, the gel, ointment, lotion, or cream formulations include:
about 0.01 to about 2.5% w/w tropane alkaloid or a pharmaceutically acceptable salt thereof; and
about 0.002% w/w to 0.5% w/w of one or more preservatives.

Thus, in some embodiments, the gel, ointment, lotion, or cream formulations include:
about 0.01 to about 2.5% w/w tropane alkaloid or a pharmaceutically acceptable salt thereof;
not more than about 0.002% w/w of one or more preservatives;
a first pharmaceutically acceptable carrier, which is a copolymer of acrylamide and sodium acryloyldimethyl taurate, isohexadecane, and polysorbate 80, which is present in about 0.5 to about 2% w/w;
a second pharmaceutically acceptable carrier, which is a polymer of acrylic acid cross-linked with one or more allyl ether of a polyalcohol, which is present in about 1 to about 3% w/w; and
a buffer having a pKₐ of between about 2.5 and 7, which is present at about 0.01 to about 5% w/w; and
wherein the gel, ointment, lotion, or cream formulations optionally include about 0.05 to about 0.3% w/w of a polycyclic nucleobase or a pharmaceutically acceptable salt thereof.

In some embodiments of the methods herein, the gel, ointment, lotion, or cream formulations include:
about 0.05 to about 2.5% w/w tropane alkaloid or a pharmaceutically acceptable salt thereof;
not more than about 0.25% w/w of one or more preservatives;
a first pharmaceutically acceptable carrier, which is a copolymer of acrylamide and sodium acryloyldimethyl taurate, isohexadecane, and polysorbate 80, which is present in about 0.5 to about 2% w/w;
a second pharmaceutically acceptable carrier, which is a polymer of acrylic acid cross-linked with one or more allyl ether of a polyalcohol, which is present in about 1 to about 3% w/w; and
a buffer having a pKₐ of between about 2.5 and 7, which is present at about 0.01 to about 5% w/w; and
wherein the gel, ointment, lotion, or cream formulations optionally include about 0.01 to about 0.3% w/w of a polycyclic nucleobase or a pharmaceutically acceptable salt thereof.

In some embodiments, the gel, ointment, lotion, or cream formulations include: 0.01%, 0.03%, 0.05%, 0.08%, about 0.15, about 0.5, or about 1% w/w tropane alkaloid or a pharmaceutically acceptable salt thereof; zero or about 0.1, 0.15, 0.16, 0.17, or 0.2% w/w polycyclic nucleobase or a pharmaceutically acceptable salt thereof; about 1% w/w of a first pharmaceutically acceptable carrier, which is a copolymer of acrylamide and sodium acryloyldimethyl taurate, isohexadecane, and polysorbate 80; about 2% w/w of a second pharmaceutically acceptable carrier, which is a polymer of acrylic acid cross-linked with one or more allyl ether of a polyalcohol; and a pH of about 4.5 to about 5.0 (e.g., about 4.7 to about 4.8, e.g., about 4.75).

In some embodiments, the gel, ointment, lotion, or cream formulations include: about 0.01%, 0.03%, 0.05%, 0.08%, or 0.15% w/w atropine sulfate; about 0.16% w/w caffeine; about 1% w/w of a first pharmaceutically acceptable carrier, which is a copolymer of acrylamide and sodium acryloyldimethyl taurate, isohexadecane, and polysorbate 80; about 2% w/w of a second pharmaceutically acceptable carrier, which is a polymer of acrylic acid cross-linked with one or more allyl ether of a polyalcohol; and a pH of about 4.5 to about 5.0 (e.g., about 4.7 to about 4.8, e.g., about 4.75).

In some embodiments, the gel, ointment, lotion, or cream formulations include: about 1% w/w atropine sulfate; about 0.16% w/w caffeine; about 1% w/w of a first pharmaceutically acceptable carrier, which is a copolymer of acrylamide and sodium acryloyldimethyl taurate, isohexadecane, and polysorbate 80; about 2% w/w of a second pharmaceutically acceptable carrier, which is a polymer of acrylic acid cross-linked with one or more allyl ether of a polyalcohol; and a pH of about 4.5 to about 5.0 (e.g., about 4.7 to about 4.8, e.g., about 4.75).

In some embodiments, the gel, ointment, lotion, or cream formulations include: about 0.15% w/w atropine sulfate; zero % w/w polycyclic nucleobase or a pharmaceutically acceptable salt thereof (e.g., caffeine); about 1% w/w of a first pharmaceutically acceptable carrier, which is a copolymer of acrylamide and sodium acryloyldimethyl taurate, isohexadecane, and polysorbate 80; about 2% w/w of a second pharmaceutically acceptable carrier, which is a polymer of acrylic acid cross-linked with one or more allyl ether of a polyalcohol; and a pH of about 4.5 to about 5.0 (e.g., about 4.7 to about 4.8, e.g., about 4.75).

In some embodiments, the gel, ointment, lotion, or cream formulations include: about 0.5% w/w atropine sulfate; zero % w/w polycyclic nucleobase or a pharmaceutically acceptable salt thereof (e.g., caffeine); about 1% w/w of a first pharmaceutically acceptable carrier, which is a copolymer of acrylamide and sodium acryloyldimethyl taurate, isohexadecane, and polysorbate 80; about 2% w/w of a second pharmaceutically acceptable carrier, which is a polymer of acrylic acid cross-linked with one or more allyl ether of a polyalcohol; and a pH of about 4.5 to about 5.0 (e.g., about 4.7 to about 4.8, e.g., about 4.75).

In some embodiments, the gel, ointment, lotion, or cream formulations include: about 0.5% w/w atropine sulfate; zero % w/w polycyclic nucleobase or a pharmaceutically acceptable salt thereof (e.g., caffeine); zero % w/w parabens; about 1% w/w of a first pharmaceutically acceptable carrier, which is a copolymer of acrylamide and sodium acryloyldimethyl taurate, isohexadecane, and polysorbate 80; about 2% w/w of a second pharmaceutically acceptable carrier, which is a polymer of acrylic acid cross-linked with one or more allyl ether of a polyalcohol; and a pH of about 4.5 to about 5.0 (e.g., about 4.7 to about 4.8, e.g., about 4.75).

In some embodiments, the gel, ointment, lotion, or cream formulations include about 5 to about 7% w/w (e.g., about 6% w/w) of a sugar alcohol. In some embodiments, the sugar alcohol is a sorbitol.

In some embodiments, the gel, ointment, lotion, or cream formulations include about 1 to about 3% w/w (e.g., about 2% w/w) of a first nonionic surfactant. In some embodiments, the first nonionic surfactant is a polysorbate 80.

In some embodiments, the gel, ointment, lotion, or cream formulations include about 0.01 to about 0.5% w/w (e.g., about 0.2% w/w) of a metal chelator. In some embodiments, the metal chelator is an ethylene diamine tetra-acetic acid (EDTA).

In some embodiments, provided herein is a gel, ointment, lotion, or cream formulation, comprising an active pharmaceutical ingredient comprising a tropanyl group (e.g., atropine), pilocarpine, travoprost, donepezil, or physostigmine.

In some embodiments, the gel, ointment, lotion, or cream formulation comprises a water content of about 20% to about 31% w/w. In some embodiments, the gel, ointment, lotion, or cream formulation comprises a water content of about 20% to about 25% w/w. In some embodiments, the gel, ointment, lotion, or cream formulation comprises a water content of no more than about 31% w/w. In some embodiments, the gel, ointment, lotion, or cream formulation comprises a water content of at least about 15% w/w.

In some embodiments, the gel, ointment, lotion, or cream formulation has a pH of about 4.0 to about 5.5. In some embodiments, the gel, ointment, lotion, or cream formulation has a pH of about 4.7.

In some embodiments, the gel, ointment, lotion, or cream formulation comprises a viscosity modifier selected from: a copolymer of acrylamide and sodium acryloyldimethyl taurate, isohexadecane, and polysorbate 80 (SEPINEO® P600); or a crosslinked polyacrylic acid (a carbomer, e.g., CARBOPOL® 980); or a combination thereof.

In some embodiments, the gel, ointment, lotion, or cream formulation comprises at least one antioxidant and at least one preservative. In some embodiments, the at least one antioxidant is selected from parabens (e.g., methyl paraben, propyl paraben), bisbiguanides (e.g., alexidine), or polyquarterium 1, or a combination thereof. In some embodiments, the at least one antioxidant is selected from butylated hydroxyanisole (BHA), ascorbic acid palmitate, or a combination thereof.

In some embodiments, the gel, ointment, lotion, or cream formulation comprises
a cosolvent selected from diethylene glycol monoethyl ether (Transcutol®) or propylene glycol, or a combination thereof, in an amount of about 1% to about 5% w/w;
an emulsifier selected from polyoxyl 35 castor oil, polysorbate 80, PEG 8000, or cetyl alcohol, or a combination thereof, in an amount of about 8.5% to about 10% w/w;
mineral oil in an amount of about 8% to about 12% w/w;
glycerin in an amount of about 4% to about 8% w/w;
at least one preservative in an amount of about 0.01 to about 0.05% w/w; and
at least one antioxidant in an amount of about 0.02% to about 0.06% w/w.

In some embodiments, the gel, ointment, lotion, or cream formulations include about 1 to about 3% w/w (e.g., about 2% w/w) of a polyether. In some embodiments, the polyether is a polyethylene glycol (PEG). In some embodiments, the polyether or PEG has an average molar mass of about at least 2,000, 4,000, 6,000, 8,000, 10,000, or 20,000 g/mol. In some embodiments, the PEG is a PEG 8000.

In some embodiments, the gel, ointment, lotion, or cream formulations include about 3 to about 6% w/w (e.g., about 4 to about 5% w/w, e.g., about 4.4% w/w) of a diol. In some embodiments, the diol has a molar mass of about 200 g/mol or less. In some embodiments, the diol is propylene glycol.

In some embodiments, the gel, ointment, lotion, or cream formulations include about 5 to about 15% w/w (e.g., about 8 to about 12% w/w, e.g., about 9, 10, or 11% w/w) of an oil. In some embodiments, the oil is a mineral oil.

In some embodiments, the gel, ointment, lotion, or cream formulations include about 3 to about 7% w/w (e.g., about 4 to about 6% w/w, e.g., about 5% w/w) of a second nonionic surfactant. In some embodiments, the second nonionic surfactant is a pegylated castor oil (e.g., the second nonionic surfactant comprises a triester of glycerol and ricinoleic acid that is pegylated). In some embodiments, the second nonionic surfactant is polyoxyl 35 castor oil.

In some embodiments, the gel, ointment, lotion, or cream formulations include about 0.3 to about 0.7% w/w (e.g., about 0.4 to about 0.6% w/w, e.g., about 0.5% w/w) of a fatty alcohol. In some embodiments, the fatty alcohol is a C₄₋₃₀ alcohol, e.g., C₆₋₂₄ alcohol, e.g., C₁₆₋₁₈ alcohol. In some embodiments the fatty alcohol is a C₄₋₃₀ alkyl-OH compound or a C₄₋₃₀ alkenyl-OH compound, e.g., C₆₋₂₄ alkyl-OH, e.g., C₁₆₋₁₈ alkyl-OH, or C₆₋₂₄ alkenyl-OH, e.g., C₁₆₋₁₈ alkenyl-OH. In some embodiments, the fatty alcohol is cetyl alcohol.

In some embodiments, the gel, ointment, lotion, or cream formulation comprises cream formulation 1, 2, 3, 4, or 5, as shown in Table 1 of Example 1.

In some embodiments, the cream formulation comprises cream formulation 6, 7, 8, 9, 10, or 11, as shown in Table 3 of Example 4. In some embodiments, the cream formulation comprises cream formulation 12, 13, or 14, as shown in Table 4 of Example 6. In some embodiments, the cream formulation comprises cream formulation 17, 18, 19, 20, or 21, as shown in Table 7 of Example 6. In some embodiments, the cream formulation comprises cream PHY012, PHY038, PHY039, PHY040, PHY041, PHY042, PHY048, PHY049, PHY050 or PHY052, as shown in Table 14 of Example 13.

In some embodiments, the gels, lotions, or creams provided herein may comprise not more than about 5.0% w/w of a tropic acid, not more than about 0.2% w/w of a 7-hydroxyhyoscyamine, not more than about 0.2% w/w of a scopolamine, not more than about 0.2% w/w of a 6-hydroxyhyoscyamine, not more than about 0.3% w/w of a hyoscyamine related compound A (a norhyoscyamine), not more than about 0.5% w/w of a littorine, or not more than about 0.2% w/w of an apoatropine, or a combination thereof.

In some embodiments, the gels, lotions, or creams provided herein comprise an apparent viscosity of about 50 k to about 200 k centipoise (cps).

Provided herein is a method of making gels, lotions, or creams comprising an active pharmaceutical agent, the method comprising
1) preparing an aqueous phase comprising water, polysorbate 80, and a copolymer of acrylamide and sodium acryloyldimethyl taurate, isohexadecane, and polysorbate 80 (e.g., SEPINEO® P600);
2) adding a crosslinked polyacrylic acid (e.g., a carbomer) to the aqueous phase mixture of step 1);
3) preparing an oil phase comprising PEG 8000, mineral oil, polyoxyl 35 castor oil, cetyl alcohol, BHA, methyl paraben, and propyl paraben;
4) mixing the oil phase of step 3) with the aqueous phase mixture of step 2) and homogenizing the mixture;
5) adjusting the pH of the homogenized mixture of step 4) to a pH of about 4.7;
6) preparing a solution phase comprising diethylene glycol monoethyl ether, propylene glycol, glycerin, and ascorbyl palmitate;
7) adding the active pharmaceutical agent to the solution phase of step 6);
8) adjusting the pH of the solution phase of step 7) to a pH of about 4.7;
9) mixing and homogenizing the solution phase of step 8) with the homogenized mixture of step 5) to obtain the gel, ointment, lotion, or cream formulation comprising physostigmine.

In some embodiments, the active pharmaceutical agent is selected from atropine, physostigmine, donepezil, pilocarpine, or travoprost.

### Methods

The gel, ointment, lotion, or cream formulations described herein are useful in delivering therapeutically relevant amounts of one or more tropane alkaloids or pharmaceutically acceptable salts thereof through the skin of a subject to an underlying tissue, structure, or organ in the subject. Thus, provided herein are methods of delivering one or more tropane alkaloids or pharmaceutically acceptable salts thereof to a subject in need thereof, comprising topically administering an effective amount of a gel, ointment, lotion, or cream formulation described herein to the subject.

Also provided herein are methods of treating one or more eye diseases by delivering therapeutically relevant amounts of one or more tropane alkaloids through the skin, e.g., at least one eyelid, of a subject suffering from one or more eye diseases. Accordingly, in some embodiments, provided herein are methods of treating an eye disease in a subject in need thereof, comprising topically administering a therapeutically effective amount of a tropane alkaloid provided herein to the subject. In some embodiments, the eye diseases treated include one or more of age-related macular degeneration, allergic conjunctivitis, blepharitis, chorioretinitis, diabetic macular edema, diabetic retinopathy, dry eye disease, episcleritis, geographic atrophy, glaucoma, graft versus host disease, inflammation due to gene therapy vectors, injury-related ocular inflammation or dry eye syndrome, iritis, keratitis, keratoconjunctivitis sicca, macular degeneration (wet or dry), meibomian gland dysfunction, myopia, non-infectious uveitis, ocular hyperemia, presbyopia (dry eye), primary or secondary Sjögren's syndrome, redness, retinal inflammation, retinal vein occlusion, sterile conjunctivitis, Thygeson superficial punctate keratitis, uveitis or Demodex blepharitis.

In some embodiments, provided herein are methods of improving delivery of a tropane alkaloid to a target tissue in a subject in need thereof, comprising topically administering a dosage of a gel, ointment, lotion, or cream formulation provided herein comprising the tropane alkaloid to a portion of skin on the subject, wherein the improved delivery is at least about 50, 100, 200, 300, 400, 500, or 1000% improved as compared to a dosage of a topically administered eye drop formulation comprising the same molar amount of the tropane alkaloid in the gel, ointment, lotion, or cream formulation. In some embodiments, the improved delivery is a target tissue concentration of the tropane alkaloid at least about 50% greater than that of a dosage of a topically administered eye drop formulation comprising the same molar amount of the tropane alkaloid in the gel, ointment, lotion, or cream formulation. In some embodiments, the portion of skin is that described throughout the present disclosure. In some embodiments, the target tissue is that described throughout the present disclosure. In some embodiments, the portion of skin is a portion of an eye lid, and the target tissue is the aqueous humor of an eye under the eye lid.

In some embodiments of the methods herein, the gel, ointment, lotion, or cream formulation includes a tropane alkaloid or a pharmaceutically acceptable salt thereof. In some embodiments of the methods herein, the gel, ointment, lotion, or cream formulation includes a tropane alkaloid or a pharmaceutically acceptable salt thereof and a polycyclic nucleobase or a pharmaceutically acceptable salt thereof.

The gels, lotions, or creams herein may be applied topically to a portion of a subject's skin. In some embodiments, the topical application is to a portion of a hand, a foot, face, neck, nose, or an eyelid (e.g., an upper eyelid or a lower eyelid, a combination thereof on one or both eyes of the subject). The gel, ointment, lotion, or cream formulations provided herein allow for the one or more tropane alkaloid or pharmaceutically acceptable salt thereof contained therein to penetrate the skin, and thereby deliver the one or more tropane alkaloid or pharmaceutically acceptable salt thereof to a tissue of the subject, including an organ or an internal tissue or region of the subject. In some embodiments, the gel, ointment, lotion, or cream formulations herein deliver a tropane alkaloid or pharmaceutically acceptable salt thereof therein to an eye of the subject following application of the formulation to the subject's skin.

In some embodiments of the methods herein, the gel, ointment, lotion, or cream formulations include:
about 0.01 to about 2.5% w/w tropane alkaloid or a pharmaceutically acceptable salt thereof;
not more than about 0.002% w/w of one or more preservative;
a first pharmaceutically acceptable carrier, which is a copolymer of acrylamide and sodium acryloyldimethyl taurate, isohexadecane, and polysorbate 80, which is present in about 0.5 to about 2% w/w;
a second pharmaceutically acceptable carrier, which is a polymer of acrylic acid cross-linked with one or more allyl ether of a polyalcohol, which is present in about 1 to about 3% w/w; and
a buffer having a pKₐ of between about 2.5 and 7, which is present at about 0.01 to about 5% w/w; and
wherein the gel, ointment, lotion, or cream formulations optionally include about 0.01 to about 0.3% w/w of a polycyclic nucleobase or a pharmaceutically acceptable salt thereof.

In some embodiments of the methods herein, the gel, ointment, lotion, or cream formulations include:
about 0.05 to about 2.5% w/w tropane alkaloid or a pharmaceutically acceptable salt thereof;
not more than about 0.002% w/w of one or more preservative;
a first pharmaceutically acceptable carrier, which is a copolymer of acrylamide and sodium acryloyldimethyl taurate, isohexadecane, and polysorbate 80, which is present in about 0.5 to about 2% w/w;
a second pharmaceutically acceptable carrier, which is a polymer of acrylic acid cross-linked with one or more allyl ether of a polyalcohol, which is present in about 1 to about 3% w/w; and
a buffer having a pKₐ of between about 2.5 and 7, which is present at about 0.01 to about 5% w/w; and
wherein the gel, ointment, lotion, or cream formulations optionally include about 0.01 to about 0.3% w/w of a polycyclic nucleobase or a pharmaceutically acceptable salt thereof.

In some embodiments of the methods herein, the gel, ointment, lotion, or cream formulations include:
about 0.01 to about 2.5% w/w tropane alkaloid or a pharmaceutically acceptable salt thereof;
not more than about 0.25% w/w of one or more preservatives;
a first pharmaceutically acceptable carrier, which is a copolymer of acrylamide and sodium acryloyldimethyl taurate, isohexadecane, and polysorbate 80, which is present in about 0.5 to about 2% w/w;
a second pharmaceutically acceptable carrier, which is a polymer of acrylic acid cross-linked with one or more allyl ether of a polyalcohol, which is present in about 1 to about 3% w/w; and
a buffer having a pKₐ of between about 2.5 and 7, which is present at about 0.01 to about 5% w/w; and
wherein the gel, ointment, lotion, or cream formulations optionally include about 0.05 to about 0.3% w/w of a polycyclic nucleobase or a pharmaceutically acceptable salt thereof.

In some embodiments of the methods herein, the cream formulation comprises a cream formulation as shown throughout the Examples below, e.g., cream formulation 1, 2, 3, 4, or 5, as shown in Table 1 of Example 1.

### Kits

In some embodiments, provided herein are packaged gels, lotions, or creams, comprising a container holding a therapeutically effective amount of at least one gel, ointment, lotion, or cream described herein, and packaging components, optionally including instructions for using the at least one gel, ointment, lotion, or cream in accordance with one or more of the methods provided herein. Such packaged components may be referred to as a kit, or as a container closure system.

The present gels, lotions, or creams and associated materials, e.g., packaged components, such as in a container closure system, can be finished as a commercial product by the usual steps performed in the present field, for example by appropriate sterilization and packaging steps. For example, the material can be treated by UV/vis irradiation (200-500 nm), for example using photo-initiators with different absorption wavelengths (e.g., Irgacure 184, 2959, e.g., 1-[4-(2-hydroxyethoxy)-phenyl]-2-hydroxy-2-methyl-1-propane-1-one), preferably water-soluble initiators (e.g. Irgacure 2959). Such irradiation is usually performed for an irradiation time of 1-60 min, but longer irradiation times may be applied, depending on the specific method. The material according to the present disclosure can be finally sterile-wrapped so as to retain sterility until use and packaged (e.g., by the addition of specific product information leaflets) into suitable containers (boxes, etc.).

According to further embodiments, the present gels, lotions, or creams can also be provided in kit form combined with other packaging components necessary for administration of the gel, ointment, lotion, or cream to the patient. For example, disclosed kits, such as for use in the treatment of an eye disease, can further comprise, for example, administration materials including spatulas, rulers, and the like along with instructions.

The kits are designed in various forms based on the specific deficiencies they are designed to treat.

The gels, lotions, or creams provided herein may be prepared and placed in a container for storage at ambient or elevated temperature. When the gels, lotions, or creams are stored in a polyolefin plastic container as compared to a polyvinyl chloride plastic container, discoloration of the formulations may be reduced. Without wishing to be bound by theory, the container may reduce exposure of the container's contents to electromagnetic radiation, whether visible light (e.g., having a wavelength of about 380-780 nm) or ultraviolet (UV) light (e.g., having a wavelength of about 190-320 nm (UV B light) or about 320-380 nm (UV A light)). Some containers also include the capacity to reduce adherence or adsorption of the active agent to the surface of the container. Some containers also include the capacity to reduce exposure of the container's contents to infrared light, or a second component with such a capacity. The containers that may be used include those made from a polyolefin such as polyethylene, polypropylene, polyethylene terephthalate, polycarbonate, polymethyl pentene, polybutene, or a combination thereof, especially polyethylene, polypropylene, or a combination thereof. In some embodiments, the container is a glass container, and the user scoops the contents out of the container with a measured spatula, scoop, or the like. In some embodiments the container is malleable, which permits the user to squeeze the contents out of the container. The container may further be disposed within a second container, for example, a paper, cardboard, paperboard, metallic film, or foil, or a combination thereof, container to further reduce exposure of the container's contents to UV, visible, or infrared light. The formulations provided herein benefit from reduced discoloration, decomposition, or both during storage in such containers. The formulations provided herein may need storage lasting up to, or longer than, three months; in some cases up to, or longer than, one year. The containers may be in any form suitable to contain the contents; for example, a bag, a bottle, a tube, or a box.

The following examples further illustrate aspects of the present disclosure. However, they are in no way a limitation of the teachings or disclosure as described herein.

### EXAMPLES

### Example 1: Cream Formulations and Processes

Certain examples of creams described herein were prepared as described in Table 1. The formulation was compounded according to the following steps: (a) the aqueous compositions, including sorbitol, polysorbate 80, SEPINEO P600®, EDTA, caffeine, and carbomer, were weighed and dissolved in citrate buffer, (b) the oil and glycol compositions, including mineral oil, castor oil, propylene glycol, PEG 8000, cetyl alcohol, alexidine, and PQ-1, were weighed and mixed, (c) Atropine sulfate was weighed and dissolved in buffer, (d) oil/glycol mixture was added to aqueous phase mixture, and mixed until homogeneous mixture was obtained, (e) Atropine sulfate solution was added to the above mixture, mixed until a homogeneous formulation obtained subsequently.

TABLE 1

| | Cream Formulation # | | | | |
|---|---|---|---|---|---|
| Ingredient (% w/w) | 1 | 2 | 3 | 4 | 5 |
| Atropine Sulfate * | 0.15 | 1 | 0.15 | 0.5 | 0.5 |
| Water | 66.39 | 65.54 | 66.35 | 66.0 | 66.2 |
| Potassium citrate | 0.20 | 0.2 | 0.2 | 0.2 | 0.2 |
| Sorbitol USP | 6 | 6 | 6 | 6 | 6 |
| Polysorbate 80 NF | 2 | 2 | 2 | 2 | 2 |
| SEPINEO P600 ® | 1 | 1 | 1 | 1 | 1 |
| Carbomer Homopolymer NF | 2 | 2 | 2 | 2 | 2 |
| EDTA | 0.2 | 0.2 | 0.2 | 0.2 | 0.2 |
| Caffeine | 0.16 | 0.16 | 0 | 0 | 0 |
| Methyl Paraben | 0 | 0 | 0.18 | 0.18 | 0 |
| Propyl Paraben | 0 | 0 | 0.02 | 0.02 | 0 |
| Alexidine, dihydrochloride | 0.0005 | 0.0005 | 0 | 0 | 0.0005 |
| PQ-1 (Polidronium chloride) | 0.001 | 0.001 | 0 | 0 | 0.001 |
| PEG 8000 NF (1.75%) | 2 | 2 | 2 | 2 | 2 |
| Propylene glycol USP | 4.4 | 4.4 | 4.4 | 4.4 | 4.4 |
| Mineral oil USP | 10 | 10 | 10 | 10 | 10 |
| Polyoxyl 35 castor oil NF | 5 | 5 | 5 | 5 | 5 |
| Cetyl alcohol USP (1%) | 0.5 | 0.5 | 0.5 | 0.5 | 0.5 |
| Total | 100 | 100 | 100 | 100 | 100 |

* Concentration calculated based on free base form.

### Example 2: Tropane Alkaloid Stability in Cream Formulations

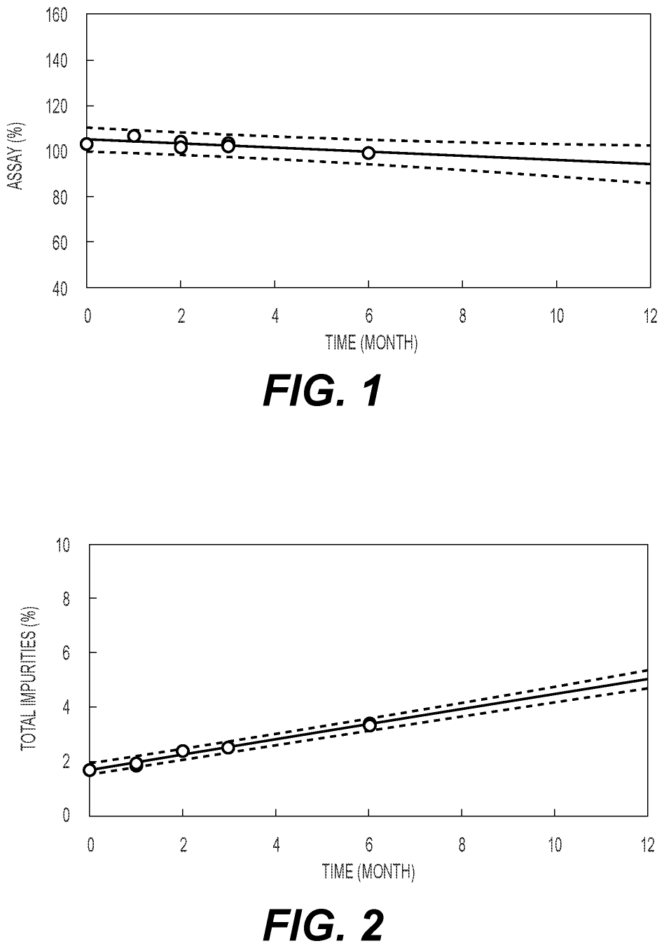

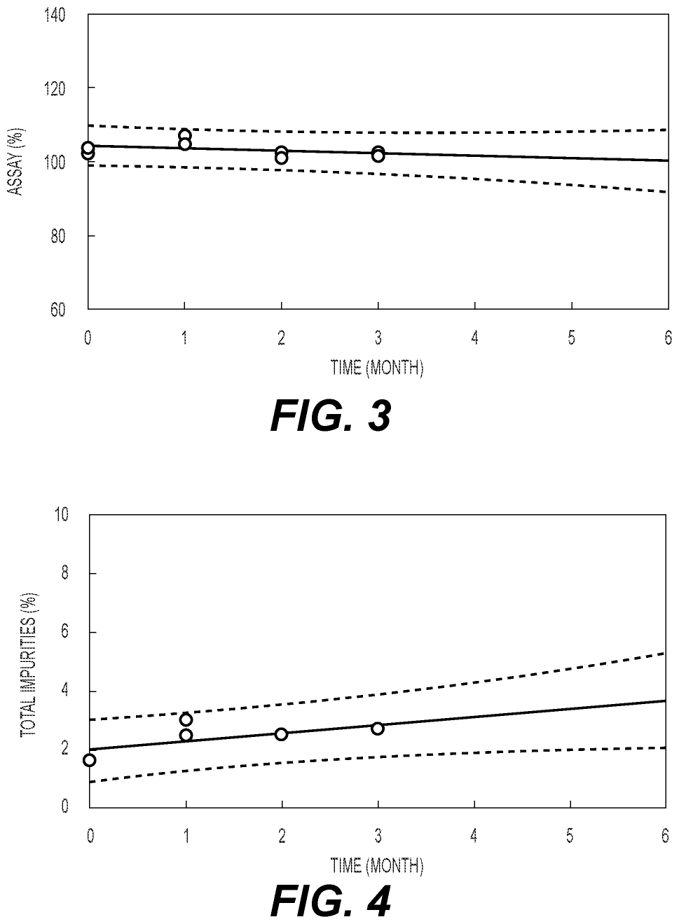

Cream formulations 1 and 2 of Table 1 were assayed for stability by exposure to 25 kiloGray (kGy) eBeam sterilization followed by storage at 40° C. in 75% relative humidity for three months, which results are shown in FIGS. 1 and 2 (the same sample of cream formulation 1 was used to generate both figures) and FIGS. 3 and 4 (the same sample of cream formulation 2 was used to generate both figures) regarding chemical purity of atropine. These data show that atropine did not degrade more than about 10% during the time period of the assay. Cream formulations 1 and 2 remained chemically stable for up to 3 months, and were projected to remain stable for at least 6 months under such conditions.

### Example 3: Tropane Alkaloid Chemical Stability

Base hydrolysis. About 17.5% of degradation was observed when atropine sulfate was exposed to 0.05 N NaOH at room temperature for 10 minutes. This indicates atropine sulfate is sensitive to base.

Acid hydrolysis. About 14.2% degradation was observed when atropine sulfate was exposed to 5 N HCl at room temperature for 3 days. This indicates atropine sulfate is sensitive to acid.

Oxidative degradation. About 12.7% degradation was observed when atropine sulfate was exposed to 30% H₂O₂ at room temperature for 7 days followed by reflux for 24 hours at 60° C. This indicates atropine sulfate is sensitive to oxidation.

Thermal and photolytic degradation. No significant variation was observed in both thermal and photolytic conditions. This indicates atropine sulfate is stable to heat and light.

### Example 4: Antimicrobial Effectiveness Testing (AET)

Cream formulations having the preservatives shown in Table 2 were prepared and antimicrobial effectiveness testing was performed according to USP <51>.

TABLE 2

| Cream | | |
|---|---|---|
| Formulation # | Preservative (% w/w or ppm) | AET |
| 6 | 0.18% methylparaben + 0.02% propylparaben | Pass |
| 7 | 0.135% methylparaben + 0.015% propylparaben | Pass |
| 8 | 0.09% methylparaben + 0.01% propylparaben | Pass |
| 9 | 10 ppm PQ-1 + 5 ppm alexidine | Pass |
| 10 | 5 ppm PQ-1 + 2.5 ppm alexidine | Pass |
| 11 | 100 ppm PQ-1 + 50 ppm alexidine (di-HCl)* | Pass |

TABLE 3
List of formulations with preservatives

| Ingredient | Cream Formulation # | | | | | | |
|---|---|---|---|---|---|---|---|
| (% w/w) | 6 | 7 | 8 | 9 | 10 | 11 | 14 |
| Atropine Sulfate* | 0.5 | 0.5 | 0.5 | 1 | 1 | 1 | 0.15 |
| Water | 66.03 | 66.08 | 66.13 | 65.54 | 65.54 | 65.53 | 66.22 |
| Potassium citrate | 0 | 0 | 0 | 0.2 | 0.2 | 0.2 | 0 |
| Sodium citrate | 0.17 | 0.17 | 0.17 | 0 | 0 | 0 | 0.17 |
| Sorbitol USP | 6 | 6 | 6 | 6 | 6 | 6 | 6 |
| Polysorbate 80 NF | 2 | 2 | 2 | 2 | 2 | 2 | 2 |
| SEPINEO P600 ® | 2 | 2 | 2 | 2 | 2 | 2 | 2 |
| Carbomer Homopolymer NF | 1 | 1 | 1 | 1 | 1 | 1 | 1 |
| EDTA | 0.2 | 0.2 | 0.2 | 0.2 | 0.2 | 0.2 | 0.2 |
| Caffeine | 0 | 0 | 0 | 0.16 | 0.16 | 0.16 | 0 |
| Methyl Paraben | 0.18 | 0.135 | 0.09 | 0 | 0 | 0 | 0 |
| Propyl Paraben | 0.02 | 0.015 | 0.01 | 0 | 0 | 0 | 0 |
| Alexidine, dihydrochloride | 0 | 0 | 0 | 0.0005 | 0.00025 | 0.005 | 0.0005 |
| PQ-1 (Polidronium chloride) | 0 | 0 | 0 | 0.001 | 0.0005 | 0.01 | 0.001 |
| PEG 8000 NF (1.75%) | 2 | 2 | 2 | 2 | 2 | 2 | 2 |
| Propylene glycol USP | 4.4 | 4.4 | 4.4 | 4.4 | 4.4 | 4.4 | 4.4 |
| Mineral oil USP | 10 | 10 | 10 | 10 | 10 | 10 | 10 |
| Polyoxyl 35 castor oil NF | 5 | 5 | 5 | 5 | 5 | 5 | 5 |
| Cetyl alcohol USP (1%) | 0.5 | 0.5 | 0.5 | 0.5 | 0.5 | 0.5 | 0.5 |
| α-cyano-4-hydroxycinnamic | 0 | 0 | 0 | 0 | 0 | 0 | 0.36 |
| acid | | | | | | | |
| Total | 100 | 100 | 100 | 100 | 100 | 100 | 100 |

*Concentration calculated based on free base form.

These data show that the combination of PQ-1 and alexidine is effective to inhibit microbial growth in semi-solid dosage forms over a range of preservative concentrations.

### Example 5: Tissue Penetration Pharmacokinetics

Three tropane alkaloid formulations were prepared, including a 0.15% w/w atropine topical ophthalmic cream (Formulation #1), a 1% w/w atropine topical ophthalmic cream (Formulation #2), and a 0.05% atropine topical ophthalmic eye drop solution (over-the-counter eye drop solution). The formulations were compared for their target tissue (aqueous humor) penetration.

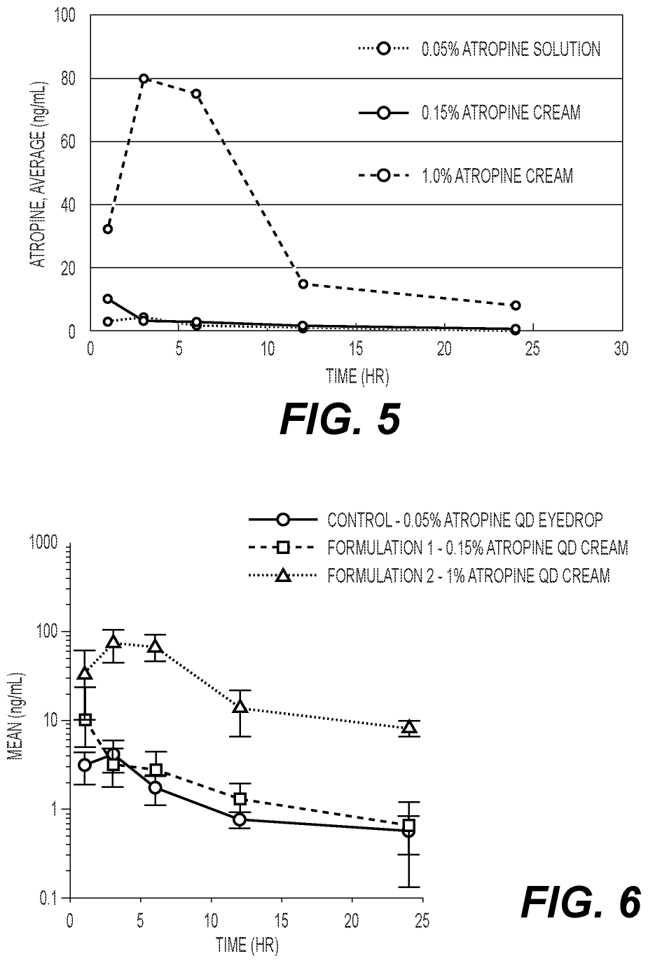

FIG. 5 and FIG. 6 (log scale of data in FIG. 5) show the average atropine concentration in the aqueous humor target tissue following single dosing (drops to the eye (corneal) or 50 mg cream formulation to the eye lid) of atropine on an equal molar basis with each formulation. These data show that the 0.15% atropine cream formulation provided target tissue (aqueous humor) exposure similar to that of the 0.05% atropine eye drop solution, indicating the cream formulations are a viable alternative to eye drops. For example, the 0.05% solution and the 0.15% cream each reached Tₘₐₓ within 1-3 hours after dosing, indicating fast absorption of the drug. There was a higher AUC₀₋₂₄ observed in the 0.15% atropine ophthalmic topical cream dosed group compared to that of 0.05% atropine eye drop dosed group, indicating a more efficient use of atropine administered.

These data also show an increase in aqueous humor exposure between 0.15% and 1% atropine ophthalmic topical cream, which indicates a smaller volume of cream could be applied instead of a larger volume of cream having lower concentration of active pharmaceutical ingredient, which could address potential patient aesthetic concerns of usage and compliance.

### Example 6: API Stabilized With Low Molecular Weight Molecules (LMW) With One or More Aromatic Ring Structures and/or Conjugated Structures

Cream formulations with and without the LMW molecule are shown in Table 4. The assay results of those formulations before and after eBeam (25 kGy) are summarized in Table 5. The API stability with LMW molecule of was successfully demonstrated. It is to be noted that cream formulations 12 and 13 were duplicate formulations to confirm reproducibility.

TABLE 4
Cream formulations with and without the LMW molecule

| | Cream Formulation # | | |
|---|---|---|---|
| Ingredient (% w/w) | 12 | 13** | 14 |
| Atropine Sulfate * | 0.15 | 0.15 | 0.15 |
| Water | 66.58 | 66.58 | 66.22 |
| Sodium citrate | 0.17 | 0.17 | 0.17 |
| Sorbitol USP | 6 | 6 | 6 |
| Polysorbate 80 NF | 2 | 2 | 2 |
| SEPINEO P600 ® | 2 | 2 | 2 |
| Carbomer Homopolymer NF | 1 | 1 | 1 |
| EDTA | 0.2 | 0.2 | 0.2 |
| Alexidine, dihydrochloride | 0.0005 | 0.0005 | 0.0005 |
| PQ-1 (Polidronium chloride) | 0.001 | 0.001 | 0.001 |
| PEG 8000 NF (1.75%) | 2 | 2 | 2 |
| Propylene glycol USP | 4.4 | 4.4 | 4.4 |
| Mineral oil USP | 10 | 10 | 10 |
| Polyoxyl 35 castor oil NF | 5 | 5 | 5 |
| Cetyl alcohol USP (1%) | 0.5 | 0.5 | 0.5 |
| α-cyano-4-hydroxycinnamic | 0 | 0 | 0.36 |
| acid | | | |
| Total | 100 | 100 | 100 |

* Concentration calculated based on free base form.
**Formulation 13 is the same as formulation 12 and demonstrates reproducibility in preparing the formulations described herein.

TABLE 5
Formulations assay with/without LMW molecule
before and after eBeam (25 kGy)

| Formulation | | | % assay change after |
|---|---|---|---|
| # | Pre-eBeam | Post eBeam | eBeam |
| 12 | 105.9% | 88.1% | −17.8% |
| 13 | 107.6% | 89.5% | −18.1% |
| 14 | 104% | 98.8% | −5.2% |

The influence of caffeine on atropine stability after eBeam process was studied. The cream formulations were compounded with and without caffeine, and the assay/total impurities were analyzed by HPLC. The test results indicated that the assay dropped 5.2% for formulation containing caffeine after eBeam, while the one without caffeine dropped 18.1% (Table 6). The total impurities increased to 1.7% for formulation containing caffeine after eBeam, while the one without caffeine increased to 8.4%. The presence of caffeine reduced the appearance of impurities under test conditions of 40° C. and 75% Relative Humidity (RH) at a time point of 3 months. The amount of impurities was about 5 times lower for formulations with caffeine compared to formulations without caffeine.

TABLE 6
Assessment of Atropine Stability With and Without Caffeine

| | | | | Assay (%) | | | | |
|---|---|---|---|---|---|---|---|---|
| | | | | | | % assay | Total | |
| | | | | | | change | Impurities (%) | |
| Atropine | Overage | Caffeine | Final | Pre- | Post- | after | Pre- | Post- |
| (% w/w) | (%) | (% w/w) | pH | eBeam | eBeam | eBeam | eBeam | eBeam |
| 0.15 | 7 | 0.16 | 4.80 | 108.1 | 102.9 | −5.2 | 0 | 1.7 |
| 0.15 | 7 | 0 | 4.64 | 107.6 | 89.5 | −18.1 | 0 | 8.4 |

The influence of a stabilizer on atropine stability after eBeam process was further investigated. The stabilizers include aromatic ring compounds (such as α-cyano-4-hydroxycinnamic acid) and hetero ring compounds (such as histidine). The cream formulations were compounded with a stabilizer (Table 7). The assay/total impurities were analyzed by HPLC, and the test results before and after the eBeam process are summarized in Table 8.

The results indicated that all the stabilizers tested herein are effective to prevent atropine from degradation when compared to the sample in Table 6 having no stabilizer.

The data showed a correlation between atropine stability and amount of α-cyano-4-hydroxycinnamic acid and histidine. The amount of tropic acid, the degradant of atropine, after eBeam process showed the same trends as in the atropine assay analysis.

TABLE 7
Formulations containing various stabilizers
in various concentrations

| Formulation # | Stabilizer (% w/w) |
|---|---|
| 17 | 0.12% α-cyano-4-hydroxycinnamic acid |
| 14 | 0.36% α-cyano-4-hydroxycinnamic acid |
| 18 | 0.24% α-cyano-4-hydroxycinnamic acid |
| 19 | 0.6% α-cyano-4-hydroxycinnamic acid |
| 20 | 0.1% L-Histidine |
| 21 | 0.5% L-Histidine |
| 22 | 0.1% Sodium Sorbate |
| 23 | 0.5% Sodium Sorbate |

TABLE 8
Assessment of stabilizers (caffeine
alternatives) on atropine Stability

| Formulation | | | % assay | | |
|---|---|---|---|---|---|
| # | Assay | Assay | change | Tropic acid | Tropic acid |
| (0.15% | (%), Pre | (%), Post | after | (%), Pre | (%), Post |
| Atropine) | eBeam | eBeam | eBeam | eBeam | eBeam |
| 17 | n/a | 86.3 | n/a | n/a | 4.2 |
| 14 | n/a | 98.8 | n/a | n/a | Not detected |
| 18 | 96.4 | 97.5 | 1.1 | Not detected | Not detected |
| 19 | 99.0 | 97.6 | −1.4 | Not detected | Not detected |
| 20 | 95.8 | 88.9 | −6.8 | Not detected | 3.7 |
| 21 | 98.6 | 100.9 | 2.3 | Not detected | 0.2 |
| 22 | 95.3 | 91.4 | −3.9 | Not detected | 3.3 |
| 23 | 96.1 | 95.0 | −1.1 | Not detected | Not detected |

TABLE 9
Cream formulations with stabilizer

| Ingredient | Cream Formulation # | | | | | | | | |
|---|---|---|---|---|---|---|---|---|---|
| (% w/w) | 15 | 16 | 17 | 18 | 19 | 20 | 21 | 22 | 23 |
| Atropine | 0.15 | 0.15 | 0.15 | 0.15 | 0.15 | 0.15 | 0.15 | 0.15 | 0.15 |
| Sulfate * | | | | | | | | | |
| Water | 66.42 | 66.58 | 66.46 | 66.14 | 65.78 | 66.28 | 65.88 | 66.28 | 65.88 |
| Citrate salt | 0.17 | 0.17 | 0.17 | 0.17 | 0.17 | 0.17 | 0.17 | 0.17 | 0.17 |
| Sorbitol USP | 6 | 6 | 6 | 6 | 6 | 6 | 6 | 6 | 6 |
| Polysorbate 80 | 2 | 2 | 2 | 2 | 2 | 2 | 2 | 2 | 2 |
| NF | | | | | | | | | |
| SEPINEO P600 ® | 2 | 2 | 2 | 2 | 2 | 2 | 2 | 2 | 2 |
| Carbomer | 1 | 1 | 1 | 1 | 1 | 1 | 1 | 1 | 1 |
| Homopolymer NF | | | | | | | | | |
| EDTA | 0.2 | 0.2 | 0.2 | 0.2 | 0.2 | 0.2 | 0.2 | 0.2 | 0.2 |
| Alexidine, | 0.0005 | 0.0005 | 0.0005 | 0 | 0 | 0 | 0 | 0 | 0 |
| dihydrochloride | | | | | | | | | |
| PQ-1 | 0.001 | 0.001 | 0.001 | 0 | 0 | 0 | 0 | 0 | 0 |
| (Polidronium | | | | | | | | | |
| chloride) | | | | | | | | | |
| Methyl paraben | 0 | 0 | 0 | 0.18 | 0.18 | 0.18 | 0.18 | 0.18 | 0.18 |
| Propyl paraben | 0 | 0 | 0 | 0.02 | 0.02 | 0.02 | 0.02 | 0.02 | 0.02 |
| PEG 8000 NF | 2 | 2 | 2 | 2 | 2 | 2 | 2 | 2 | 2 |
| (1.75%) | | | | | | | | | |
| Propylene glycol | 4.4 | 4.4 | 4.4 | 4.4 | 4.4 | 4.4 | 4.4 | 4.4 | 4.4 |
| USP | | | | | | | | | |
| Mineral oil USP | 10 | 10 | 10 | 10 | 10 | 10 | 10 | 10 | 10 |
| Polyoxyl 35 | 5 | 5 | 5 | 5 | 5 | 5 | 5 | 5 | 5 |
| castor oil NF | | | | | | | | | |
| Cetyl alcohol | 0.5 | 0.5 | 0.5 | 0.5 | 0.5 | 0.5 | 0.5 | 0.5 | 0.5 |
| USP (1%) | | | | | | | | | |
| Caffeine | 0.16 | 0 | 0 | 0 | 0 | 0 | 0 | 0 | 0 |
| α-cyano-4- | 0 | 0 | 0.12 | 0.24 | 0.6 | 0 | 0 | 0 | 0 |
| hydroxycinnamic | | | | | | | | | |
| acid | | | | | | | | | |
| Histidine | 0 | 0 | 0 | 0 | 0 | 0.1 | 0.5 | 0 | 0 |
| Sodium Sorbate | 0 | 0 | 0 | 0 | 0 | 0 | 0 | 0.1 | 0.5 |
| Total | 100 | 100 | 100 | 100 | 100 | 100 | 100 | 100 | 100 |

* Concentration calculated based on free base form

### Example 7: Change In Pupil Diameter

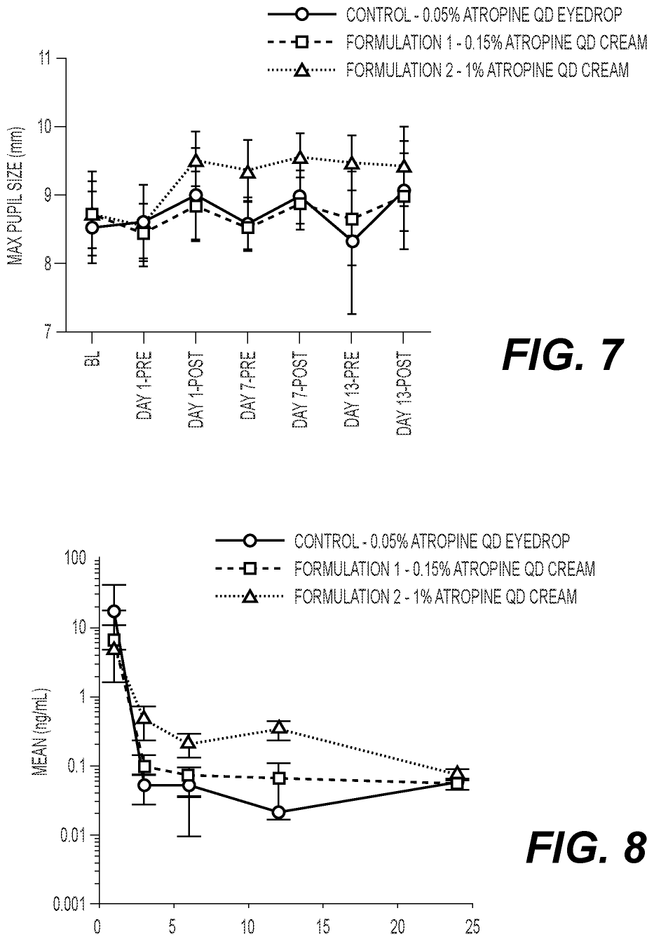

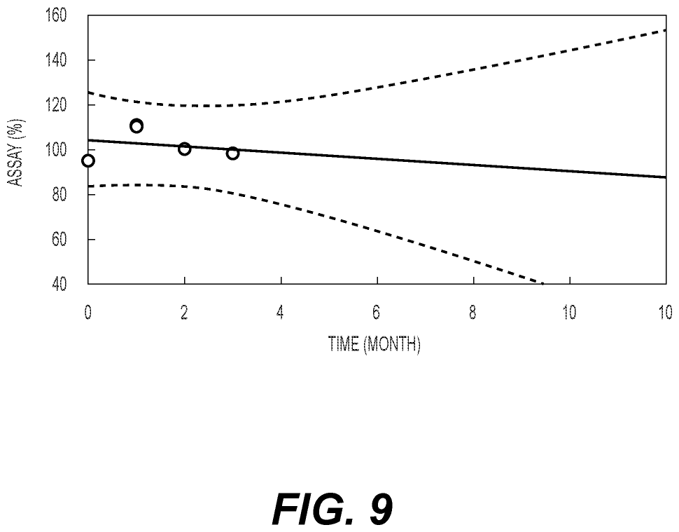

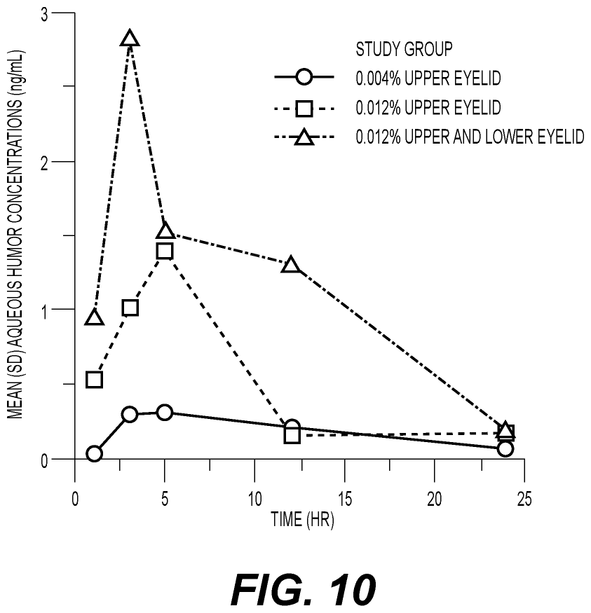

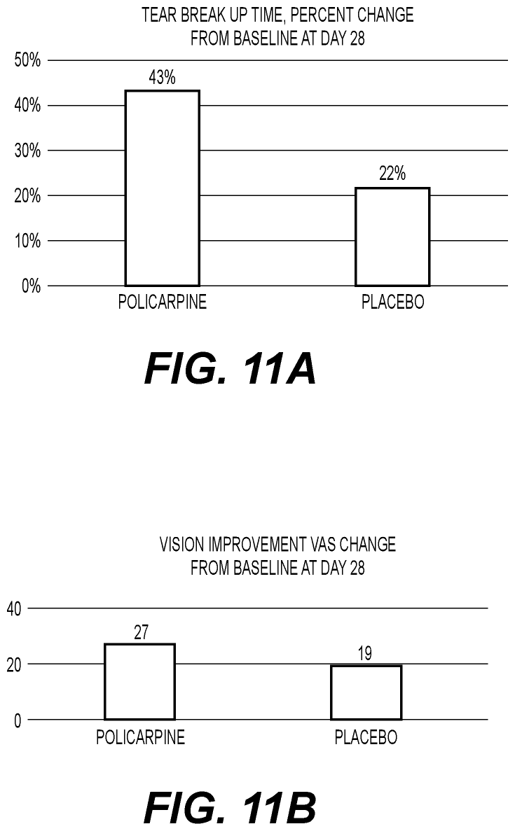

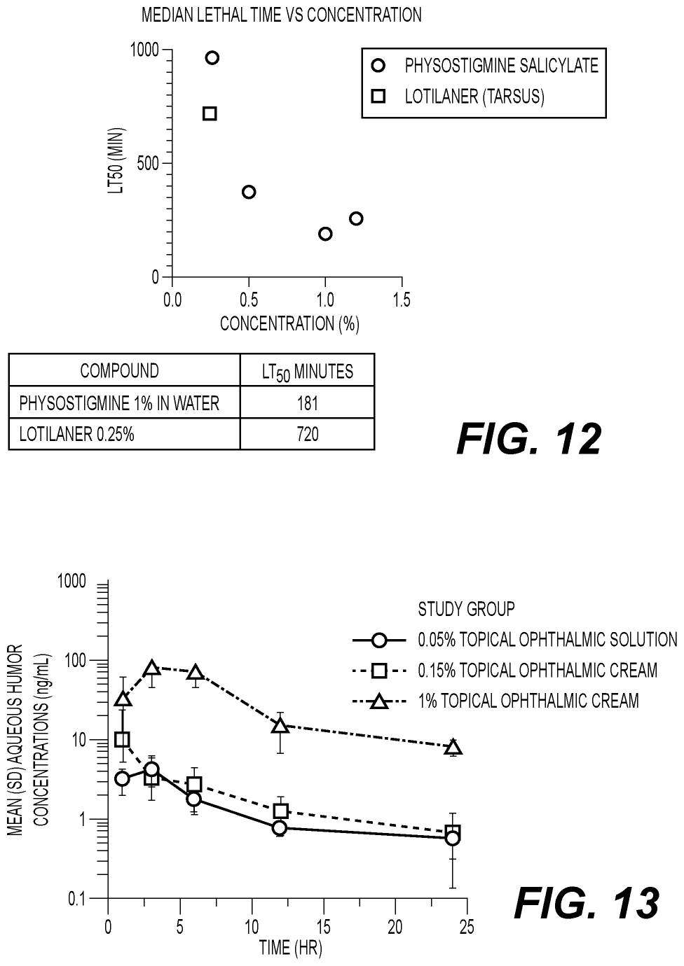

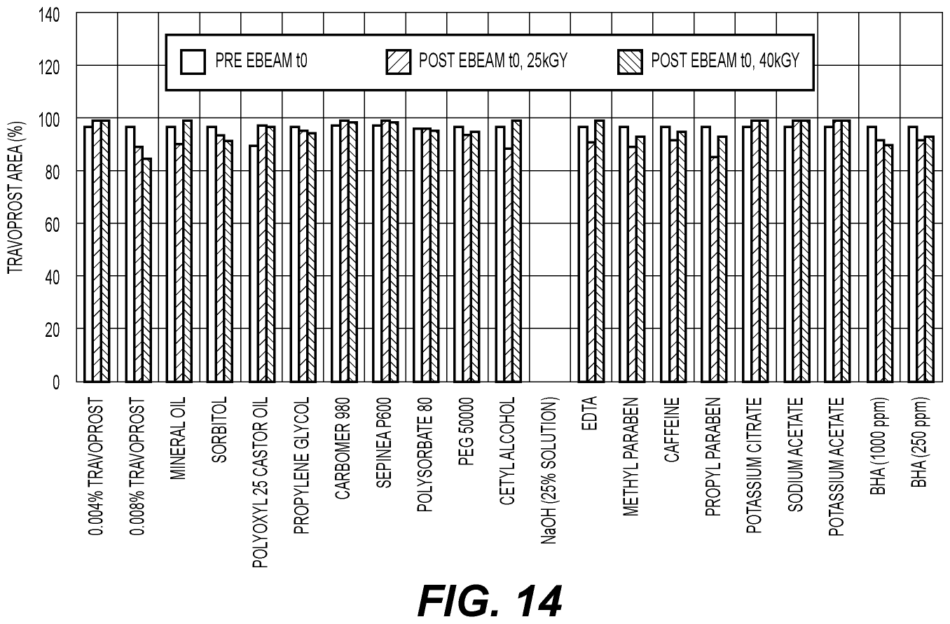

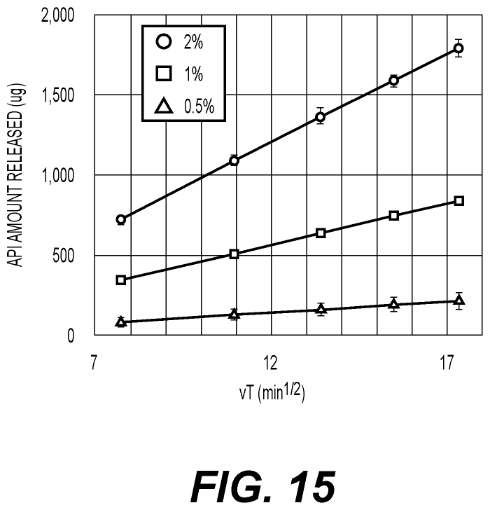

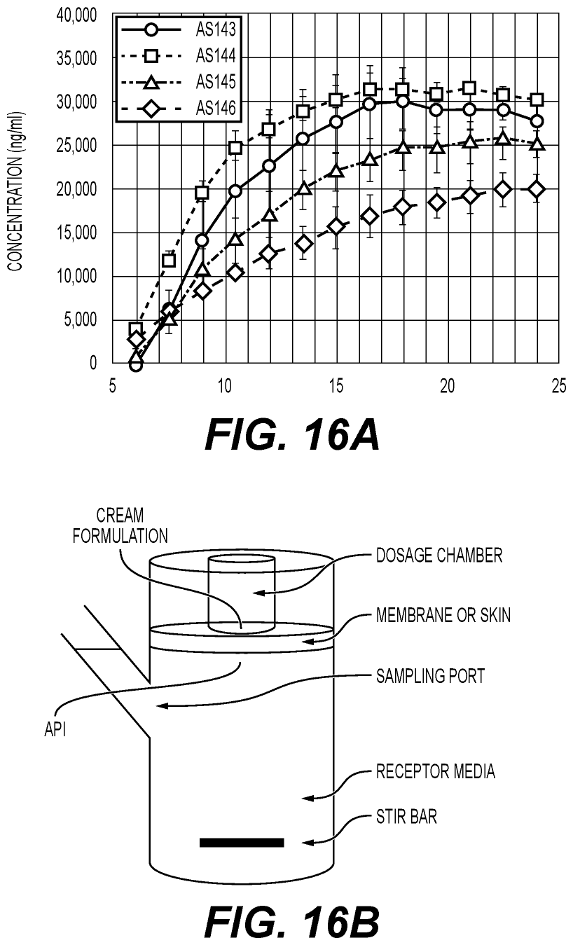

Atropine cream 0.15% was compared with atropine eye drops 0.05%. See FIG. 7. All dose levels were well-tolerated based on clinical observations, and dermal and ocular assessments. Pupil dilation observed with atropine cream and eye drops was similar (+0.5 mm). The dilation was recorded after 14-days.

### Example 8: Minipig Plasma Exposure

As shown in FIG. 8, systemic exposure with atropine topical ophthalmic cream is 100 or more times lower than systemic exposure of commercially approved atropine sulfate ophthalmic solution, USP 1%.

### Example 9: Physostigmine Formulations Water Content

Five formulations, containing 0% to 25% w/w of water, were compounded (PHY038 to PHY042). The test results as shown in Table 10 indicate that formulations with a water content of 15% or less showed phase separation under centrifugation suggesting poor physical stability of these formulations. Formulations with a water content of 20% or higher showed no phase separation under centrifugation suggesting desirable physical stability of these formulations.

TABLE 10
Forced phase separation of formulations at different water content
by centrifugation (13,500 rpm (~16,800 RCF) for 5 min)

| | Water | | |
|---|---|---|---|
| Formulation | content | | |
| # | (% w/w) | pH | 25° C./65% RH, no eBeam, 4 months |
| PHY038 | 0 | 4.45 | No forced phase separation observed. |
| PHY039 | 10 | 4.79 | Forced phase separation observed |
| PHY040 | 15 | 4.57 | Minor separation observed |
| PHY041 | 20 | 4.52 | No forced phase separation observed |
| PHY042 | 25 | 4.44 | No forced phase separation observed |
| PHY030 | 32.37 | 4.47 | No forced phase separation observed |
| | | | (25 kGy eBeam, 1 month stored at 40 |
| | | | C./75% RH) |

Formulation development with low water content (10-15%) failed the physical stability test. It was found that formulation PHY038, PHY039, and PHY040 with a water content of 0%, 10%, and 15%, respectively, showed a phase separation under a centrifugation test at 13,500 rpm (˜16,800 RCF) for 5 min.

Formulations with high water content or high pH failed in chemical stability testing. It was found that PHY030 had a physostigmine assay pre-eBeam=94.4%, after 25 kGy eBeam=85%, and the total impurity pre-eBeam=0.14%, after 25 kGy eBeam=0.58%.

### Example 10: Physostigmine Formulations Antioxidant Content

Formulations were compounded with butylated hydroxyanisole (BHA), or a combination of BHA and ascorbyl palmitate at different concentrations (PHY048 to PHY052). The results of total impurities are summarized in Table 11.

TABLE 11
Total Impurities of formulations with various concentrations
of antioxidants at 40° C./75% RH over time

| | | ascorbyl | | Pre- | Post | Total |
|---|---|---|---|---|---|---|
| | Physostigmine | palmitate | BHA | eBeam | eBeam | impurities |
| ID | (% w/w) | (% w/w) | (% w/w) | (%) | (%) | 1-month |
| PHY049 | 1 | 0 | 0.025 | 0.27 | 0.27 | 0.56 |
| | | | | | 0.32 | |
| PHY050 | 1 | 0 | 0.655 | 0.13 | 0.26 | 0.62 |
| PHY052 | 1 | 0.02 | 0.025 | 0.33 | 0.28 | 0.52 |
| | | | | | 0.28 | |

Formulations with combination of ascorbyl palmitate and BHA demonstrated lower total impurities when compared with those of BHA alone at 1 month (stored at accelerated 40° C./75% RH condition)

Formulations with BHA alone showed a little darker cream color but with acceptable impurity level at 40° C./75RH accelerated stability for 3 months when compared to formulation with both BHA and ascorbyl palmitate. In other words, under accelerated stability conditions, formulation color appears to be lighter (better aesthetics) when using both antioxidants in formulations.

### Example 11: Physostigmine Physical Characteristics

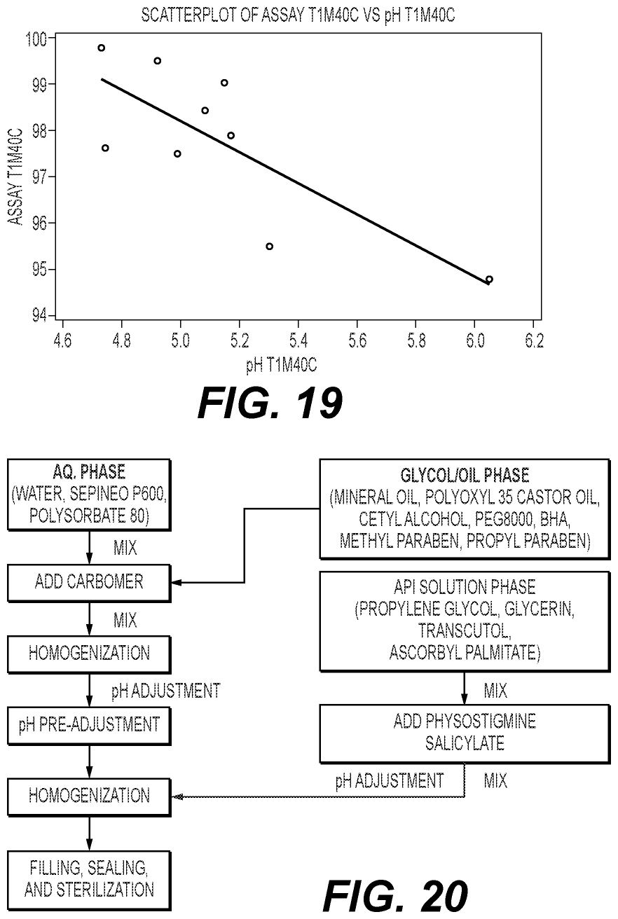

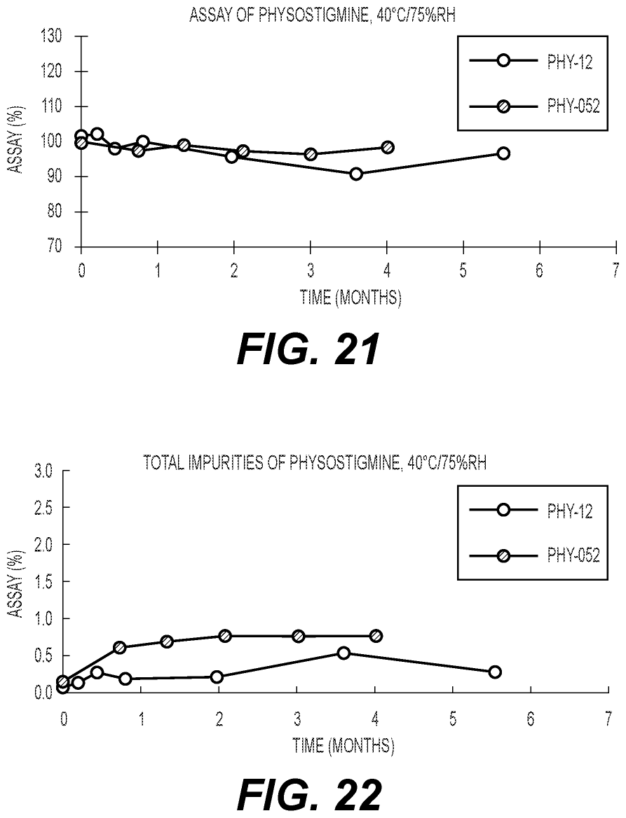

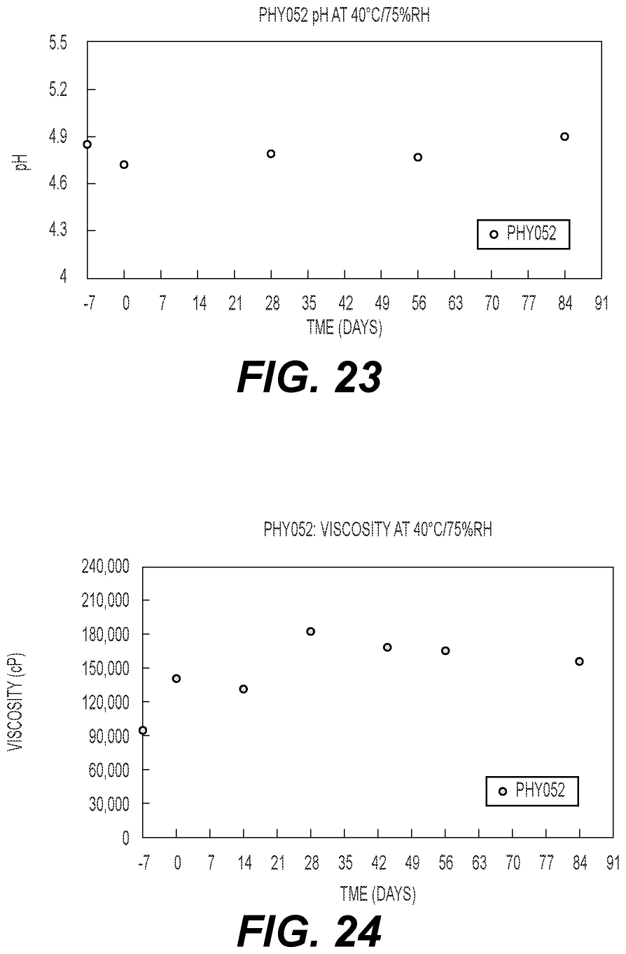

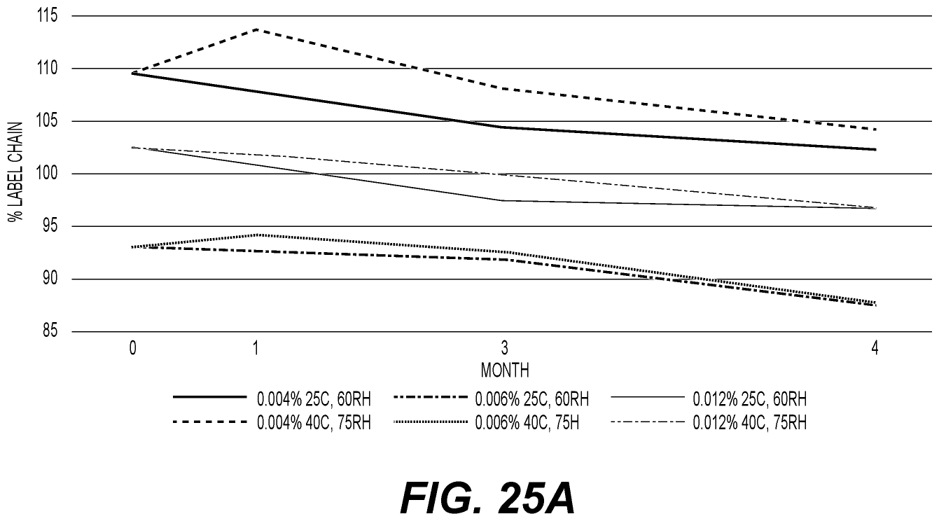

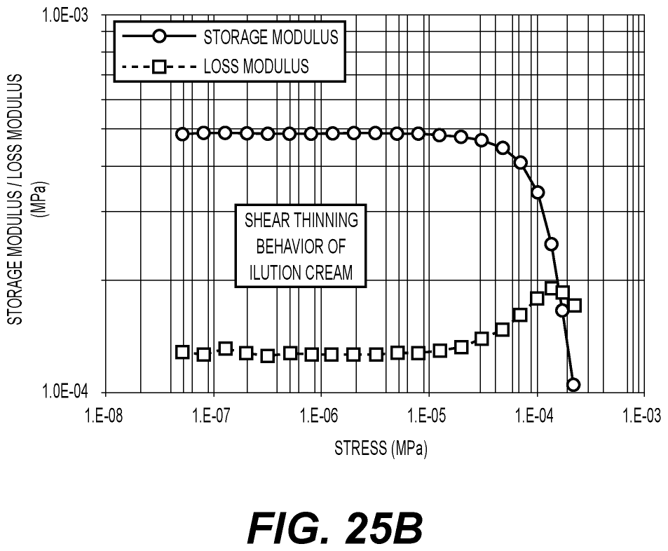

A formulation design experiment was conducted to optimize the formulation composition to achieve desirable physical properties. The experimental design is shown in Table 12 and the data analysis results are shown in FIG. 18 and FIG. 19.

TABLE 12
Design of experiment and results of formulation
thickeners and pH on cream viscosity

| Input | | | |
|---|---|---|---|
| SEPINEO P600 ® | CARBOPOL ® 980 | Output | |
| (% w/w) | (% w/w) | pH | Viscosity (cps) |
| 2 | 1.5 | 4.98 | 75060 |
| 2 | 2 | 4.90 | 112400 |
| 2 | 2 | 4.30 | 121400 |

It was found that (a) formulation viscosity increased when the CARBOPOL® 980 concentration increased, (b) formulation viscosity decreased when the pH increased, (c) no phase separation was found in the formulation comprising 2% SEPINEO P600® and 2% CARBOPOL® 980 in a centrifugation test.

### Example 12: Physostigmine Excipient Compatibility

A drug excipient compatibility (DEC) study was conducted to evaluate excipients as shown in Table 13. All excipients used in the formulations were compatible with physostigmine before and after eBeam of 25 kGy. NaOH was used to adjust the cream pH and was added before mixing with physostigmine. The chemical stability of physostigmine in formulation is stable at acidic pH environments (e.g., pH of about 3.5 to about 5.5).

TABLE 13
Drug-Excipient Compatibility - Total Impurities

| | | | Stability |
|---|---|---|---|
| | | Post | at 1 |
| | Pre | eBeam | Month, |
| Test article | eBeam | T0 | 40° C. |
| Physostigmine Salicylate | 0.11 | 0.15 | 0.15 |
| 10 mg/mL water | 0.12 | 1.64 | N/A |
| Physostigmine stock solution | 0.12 | 0.23 | 0.16 |
| Sorbitol | 0.11 | 0.20 | 0.22 |
| Polysorbate 80 | 0.11 | 0.24 | 0.21 |
| SEPINEO P600 ® | 0.11 | 0.22 | 0.26 |
| CARBOPOL ® 980 | 0.12 | 0.47 | 0.11 |
| EDTA | 0.12 | 0.26 | 0.26 |
| Mineral oil | 0.11 | 0.24 | 0.22 |
| Polyoxyl 35 castor oil | 0.12 | 0.15 | 0.16 |
| PEG 8000 | 0.11 | 0.26 | 0.17 |
| Cetyl alcohol | 0.11 | 0.25 | 0.17 |
| Alexidine | 0.11 | 0.27 | 0.22 |
| PQ-1 | 0.11 | 0.26 | 0.18 |
| Ascorbyl Palmitate | 0.11 | 0.21 | 0.23 |
| BHA | 0.11 | 0.21 | 0.17 |
| Transcutol | 0.12 | 0.17 | 0.17 |
| Propylene glycol | 0.12 | 0.26 | 0.22 |
| Glycerin | 0.11 | 0.21 | 0.26 |
| Methylparaben | 0.11 | 0.16 | N/A |
| Propylparaben | 0.11 | 0.18 | N/A |
| NaOH solution (25%) | 98.92 | 99.08 | N/A |
| Citrate buffer, pH 3.5 | 0.11 | 0.17 | 0.18 |
| Citrate buffer, pH 4 | 0.11 | 0.20 | 0.18 |
| Citrate buffer, pH 5 | 0.11 | 0.20 | 0.32 |
| Citrate buffer, pH 6 | 0.16 | 0.25 | 0.47 |
| Physostigmine Salicylate in Transcutol only | ND | 0.11 | N/A |
| Physostigmine Salicylate in Propylene | 0.16 | 0.19 | N/A |
| Glycol only | | | |

N/A means no assay was conducted.

### Example 13: Preparation of Physostigmine Formulations

The formulations comprising physostigmine were prepared according to the flowchart of FIG. 20. Table 14 provides the different formulations that were prepared.

TABLE 14
Formulations of physostigmine topical creams

| Ingredient | | | | | | | | | | | |
|---|---|---|---|---|---|---|---|---|---|---|---|
| (% w/w) | PHY012* | PHY030 | PHY038 | PHY039 | PHY040 | PHY041 | PHY042 | PHY048 | PHY049 | PHY050 | PHY052 |
| Physostigmine | 1 | 1 | 0 | 0 | 0 | 0 | 0 | 1 | 1 | 1 | 1 |
| Ascorbyl | 0.02 | 0.02 | 0.02 | 0.02 | 0.02 | 0.02 | 0.02 | 0.02 | 0 | 0 | 0.02 |
| Palmitate | | | | | | | | | | | |
| TRANS- | 14 | 14 | 14 | 14 | 14 | 14 | 14 | 14 | 14 | 14 | 14 |
| CUTOL ® | | | | | | | | | | | |
| Propylene | 22.25 | 22.25 | 56 | 48.25 | 31.11 | 43.25 | 33.25 | 34.66 | 29.68 | 29.05 | 29.66 |
| glycol | | | | | | | | | | | |
| Glycerin | 6 | 6 | 6 | 6 | 6 | 6 | 6 | 6 | 6 | 6 | 6 |
| Water, WFI | 32.5 | 32.37 | 0 | 10 | 15 | 20 | 25 | 20 | 25 | 25 | 25 |
| SEPINEO ® | 2 | 2 | 2 | 2 | 2 | 2 | 2 | 2 | 2 | 2 | 2 |
| P600 | | | | | | | | | | | |
| Polysorbate | 2 | 2 | 2 | 2 | 2 | 2 | 2 | 2 | 2 | 2 | 2 |
| 80 | | | | | | | | | | | |
| Carbomer | 2 | 2 | 2 | 2 | 2 | 2 | 2 | 2 | 2 | 2 | 2 |
| Homopolymer | | | | | | | | | | | |
| Type C | | | | | | | | | | | |
| Methylparaben | 0 | 0.18 | 0.18 | 0.18 | 0.18 | 0.18 | 0.18 | 0.18 | 0.18 | 0.18 | 0.18 |
| Polyethylene | 2 | 2 | 2 | 2 | 2 | 2 | 2 | 2 | 2 | 2 | 2 |
| glycol 8000 | | | | | | | | | | | |
| Mineral oil | 10 | 10 | 10 | 10 | 10 | 10 | 10 | 10 | 10 | 10 | 10 |
| Poly- | 5 | 5 | 5 | 5 | 5 | 5 | 5 | 5 | 5 | 5 | 5 |
| Oxyl 35 | | | | | | | | | | | |
| Castor | | | | | | | | | | | |
| oil | | | | | | | | | | | |
| Cetyl | 0.5 | 0.5 | 0.5 | 0.5 | 0.5 | 0.5 | 0.5 | 0.5 | 0.5 | 0.5 | 0.5 |
| alcohol | | | | | | | | | | | |
| EDTA | 0 | 0.05 | 0.01 | 0.01 | 0.01 | 0.01 | 0.01 | 0 | 0 | 0 | |
| BHA | 0.025 | 0.025 | 0.025 | 0.025 | 0.025 | 0.025 | 0.025 | 0.025 | 0.025 | 0.655 | 0.502 |
| Propylparaben | 0 | 0.02 | 0.02 | 0.02 | 0.02 | 0.02 | 0.02 | 0.02 | 0.02 | 0.02 | 0.02 |
| NaOH | q.s | q.s. | q.s | q.s | q.s | q.s | q.s | q.s | q.s | q.s | q.s |
| 25% | to | to | to | to | to | to | to | to | to | to | to |
| w/w | pH 4.7 | pH 4.7 | pH 4.7 | pH 4.7 | pH 4.7 | pH 4.7 | pH 4.7 | pH 4.7 | pH 4.7 | pH 4.7 | pH 4.7 |
| solution | | | | | | | | | | | |

*PHY012 contains 5 ppm of alexidine and 10 ppm of polyquarternium-1

A general procedure for preparation of a formulation of Table 14 comprises gently dissolving physostigmine in a mixture of propylene glycol and purified diethylene glycol monoethyl ether (or Transcutol) along with ascorbyl palmitate to minimize hydrolytic, oxidative, and thermal degradation during the manufacturing process. Preparing a water phase mixture, adding carbomer, preparing an oil phase mixture, mixing, homogenization, and pH adjustment as shown in FIG. 20, then adding dissolved physostigmine solution as shown in FIG. 20 to the base cream to minimize the exposure time. The procedures described herein ensure that physostigmine will be present in the oil/glycol phase instead of aqueous phase until the time of adding to the base cream.

### Example 14: Testing of Physostigmine Formulations

Six sensory attributes such as appearance, spreadability, tackiness, comfort, greasiness/oily, and residues were evaluated for three formulations. PHY052 had the best overall sensory scores. PHY048 was deemed the oiliest but still acceptable by most subjects. PHY030 was deemed the least oily among the three formulations. No skin irritation, stinging, burning, or discomfort was detected from any of the 19 subjects for all three tested formulations.

The data in the examples above, and in the Figures, demonstrates that certain physostigmine formulations are stable for at least 3 months at 40° C./75% RH, and that the formulations provide desirable release and penetration of physostigmine.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

Where references have been made to patents and printed publications throughout this specification, each of the above-cited references and printed publications are individually incorporated herein by reference in their entirety.

1.-3. (canceled)

4. A gel, ointment, lotion, or cream formulation comprising an active pharmaceutical ingredient comprising a tropanyl group, wherein the gel, ointment, lotion, or cream formulation is an oil-in-water emulsion cream formulation that includes at least 50% w/w water.

5. The gel, ointment, lotion, or cream formulation of claim 4, wherein the active pharmaceutical ingredient is a tropane alkaloid or a pharmaceutically acceptable salt thereof.

6. The cream formulation of claim 4, wherein the active pharmaceutical ingredient includes atropine, benzatropine, cocaine, homatropine, hyoscyamine, scopolamine, tiotropium, tropisetron, or trospium, or a pharmaceutically acceptable salt thereof.

7. The gel, ointment, lotion, or cream formulation of claim 4, wherein the active pharmaceutical ingredient is atropine or a pharmaceutically acceptable salt thereof, which comprises more than 50% by weight of (1R,3r,5S)-8-methyl-8-azabicyclo[3.2.1]octan-3-yl (S)-3-hydroxy-2-phenylpropanoate or a pharmaceutically acceptable salt thereof.

8. The gel, ointment, lotion, or cream formulation of claim 4, wherein the active pharmaceutical ingredient is atropine or a pharmaceutically acceptable salt thereof, which comprises less than 10% by weight of (1R,3r,5S)-8-methyl-8-azabicyclo[3.2.1]octan-3-yl (R)-3-hydroxy-2-phenylpropanoate or a pharmaceutically acceptable salt thereof.

9. The gel, ointment, lotion, or cream formulation of claim 4, wherein the active pharmaceutical ingredient is atropine or a pharmaceutically acceptable salt thereof, which comprises more than 50% by weight of (1R,3r,5S)-8-methyl-8-azabicyclo[3.2.1]octan-3-yl (R)-3-hydroxy-2-phenylpropanoate or a pharmaceutically acceptable salt thereof.

10. The gel, ointment, lotion, or cream formulation of claim 4, wherein the active pharmaceutical ingredient is atropine or a pharmaceutically acceptable salt thereof, which comprises less than 10% by weight of (1R,3r,5S)-8-methyl-8-azabicyclo[3.2.1]octan-3-yl (S)-3-hydroxy-2-phenylpropanoate or a pharmaceutically acceptable salt thereof.

11. The gel, ointment, lotion, or cream formulation of claim 4, further comprising a heterocyclic compound having the formula: 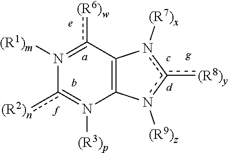
or a pharmaceutically acceptable salt thereof,
wherein
each of [symbol] a, [symbol] b, [symbol] c, [symbol] d, [symbol] e, [symbol] f, and [symbol] g is, independently, a single bond or a double bond;
each of m, n, p, w, x, y, and z is, independently, 0 or 1;
each of R¹, R³, R⁷, and R⁹ is, independently, H, C₁₋₆ alkyl, C₁₋₆ alkanolyl, C₁₋₆ haloalkyl, C₂₋₆ alkenyl, C₂₋₈ alkanalyl, C₃₋₈ alkenalyl, C₂₋₈ alkanonyl, or C₄₋₈ alkenonyl;
each of R², R⁶, and R⁸ is, independently, H, halo, O—(C₁₋₃ alkyl), NH₂, N(H) (C₁₋₃ alkyl), N(C₁₋₃ alkyl)(C₁₋₃ alkyl), S, or O.

12. The gel, ointment, lotion, or cream formulation of claim 4, further comprising a polycyclic nucleobase or a pharmaceutically acceptable salt thereof.

13. The gel, ointment, lotion, or cream formulation of claim 12, wherein the polycyclic nucleobase is azathioprine, adenine, 2-aminopurine, 2,6-diaminopurine, 2,6,8-triaminopurine, 2-amino-6-methoxy-purine, 2-chloro-6-aminopurine, 2-fluoro-6-aminopurine, guanine, thioguanine, isoguanine, purine, mercaptopurine, hypoxanthine, xanthine, 1-methylxanthine, 3-methylxanthine, 7-methylxanthine, 1,3-dimethylxanthine, 3,7-dimethylxanthine, 1,7-dimethylxanthine, 1,3,7-trimethylxanthine, 8-chloro-1,3-dimethylxanthine, uric acid, 1-methyluric acid, 3-methyluric acid, 7-methyluric acid, 1,3-dimethyluric acid, 3,7-dimethyluric acid, 1,7-dimethyluric acid, or 1,3,7-trimethyluric acid, or a pharmaceutically acceptable salt thereof.

14. The gel, ointment, lotion, or cream formulation of claim 4, comprising atropine or a pharmaceutically acceptable salt thereof and 1,3,7-trimethylxanthine.

15. The cream formulation of claim 4, comprising:
about 0.01 to about 2.5% w/w tropane alkaloid or a pharmaceutically acceptable salt thereof;
not more than about 0.25% w/w of one or more preservative;
a first pharmaceutically acceptable carrier, which is a copolymer of acrylamide and sodium acryloyldimethyl taurate, isohexadecane, and polysorbate 80, which is present in about 0.5 to about 2% w/w;
a second pharmaceutically acceptable carrier, which is a polymer of acrylic acid cross-linked with one or more allyl ether of a polyalcohol, which is present in about 1 to about 3% w/w; and
a buffer having a pKₐ of between about 2.5 and 7, which is present at about 0.01% to about 5% w/w; and
wherein the gel, ointment, lotion, or cream formulation optionally includes about 0.01 to about 0.3% w/w of a polycyclic nucleobase or a pharmaceutically acceptable salt thereof.

16. A composition, comprising:
one or more of a purified tropane alkaloid or a pharmaceutically acceptable salt thereof; and
one or more of a purified polycyclic nucleobase or a pharmaceutically acceptable salt thereof.

17. (canceled)

18. The gel, ointment, lotion, or cream formulation of claim 4, further comprising at least one low molecular weight compound or a pharmaceutically acceptable salt thereof, wherein the at least one low molecular weight compound optionally comprises one or more aromatic ring structures.

19. (canceled)

20. The gel, ointment, lotion, or cream formulation of claim 18, wherein the at least one low molecular weight compound is selected from caffeine, benzoic acid, sinapinic acid, α-cyano-4-hydroxycinnamic acid, histidine, dithranol, or a pharmaceutically acceptable salt thereof, or a combination thereof.

21. The gel, ointment, lotion, or cream formulation of claim 18, wherein the at least one low molecular weight compound is present in the cream formulation in an amount of about 0.01% to about 1% w/w.

22.-23. (canceled)

24. A gel, ointment, lotion, or cream formulation, comprising an active pharmaceutical ingredient comprising a tropanyl group, pilocarpine, travoprost, or physostigmine.

25.-30. (canceled)

31. The gel, ointment, lotion, or cream formulation of claim 24, comprising
a cosolvent selected from diethylene glycol monoethyl ether or propylene glycol, or a combination thereof, in an amount of about 1% to about 5% w/w;
an emulsifier selected from polyoxyl 35 castor oil, polysorbate 80, PEG 8000, or cetyl alcohol, or a combination thereof, in an amount of about 8.5% to about 10% w/w;
mineral oil in an amount of about 8% to about 12% w/w;
glycerin in an amount of about 4% to about 8% w/w;
at least one preservative in an amount of about 0.01 to about 0.05% w/w; and
at least one antioxidant in an amount of about 0.02% to about 0.06% w/w.

32.-35. (canceled)

36. A method of treatment, comprising administering the gel, ointment, lotion, or cream formulation of claim 4, to a subject in need thereof, wherein the subject suffers from, or is predisposed to develop, an ophthalmic condition selected from one or more of amblyopia, hyperopia, myopia, presbyopia, glaucoma, or Demodex blepharitis.

37.-40. (canceled)

41. A method of improving delivery of a tropane alkaloid to a target tissue in a subject in need thereof, comprising topically administering a dosage of the gel, ointment, lotion, or cream formulation of claim 5 comprising the tropane alkaloid to a portion of skin on the subject, wherein the improved delivery is a target tissue concentration of the tropane alkaloid at least about 50% greater than that of a dosage of a topically administered eye drop formulation comprising the same molar amount of the tropane alkaloid in the gel, ointment, lotion, or cream formulation.

42. (canceled)

43. A method for preparing the gel, ointment, lotion, or cream formulation of claim 24, the method comprising
1) preparing an aqueous phase comprising water, polysorbate 80, and a copolymer of acrylamide and sodium acryloyldimethyl taurate, isohexadecane, and polysorbate 80;
2) adding a crosslinked polyacrylic acid to the aqueous phase mixture of step 1);
3) preparing an oil phase comprising PEG 8000, mineral oil, polyoxyl 35 castor oil, cetyl alcohol, BHA, methyl paraben, and propyl paraben;
4) mixing the oil phase of step 3) with the aqueous phase mixture of step 2) and homogenizing the mixture;
5) adjusting the pH of the homogenized mixture of step 4) to a pH of about 4.7;
6) preparing a solution phase comprising diethylene glycol monoethyl ether, propylene glycol, glycerin, and ascorbyl palmitate;
7) adding an active pharmaceutical agent to the solution phase of step 6);
8) adjusting the pH of the solution phase of step 7) to a pH of about 4.7;
9) mixing and homogenizing the solution phase of step 8) with the homogenized mixture of step 5) to obtain the gel, ointment, lotion, or cream formulation of any one of claims 24-32.

44.-45. (canceled)
